(19) United States
(12) Patent Application Publication
(10) Pub. No.: US 2007/0298116 A1
(43) Pub. Date: Dec. 27, 2007

(54) Amorphous, spray-dried powders having a reduced moisture content and a high long term stability

(75) Inventors: Karoline Bechtold-Peters, Biberach-Rissegg (DE); Wolfgang Friess, Iffeldorf (DE); Stefanie Schuele, Muenchen (DE); Stefan Bassarab, Biberach (DE); Patrick Garidel, Norderstedt (DE); Torsten Schultz-Fademrecht, Biberach-Stafflangen (DE)

Correspondence Address: MICHAEL P. MORRIS;BOEHRINGER INGELHEIM CORPORATION, 900 RIDGEBURY ROAD, P. O. BOX 368, RIDGEFIELD, CT, 06877-0368, US

(73) Assignee: Boehringer Ingelheim Pharma GmbH & Co. KG, Ingelheim (DE)

(21) Appl. No.: 10/915,298

(22) Filed: Aug. 10, 2004

Related U.S. Application Data
(60) Provisional application No. 60/503,115, filed on Sep. 15, 2003.

(30) Foreign Application Priority Data
Aug. 22, 2003 (DE) 103 39 197

(51) Int. Cl. A61K 9/14; A61K 38/18; A61K 38/43; A61P 43/00; A61K 39/395

(52) U.S. Cl. 424/499; 424/130.1; 424/94.1; 514/2

(57) ABSTRACT

The present invention relates to spray-dried amorphous powders which contain a pharmaceutical active substance and a matrix-forming agent, preferably a polymer, sugar or sugar alcohol in an amount of ≧20-60% (w/w). The powders have a moisture content which is less than 1.2% (w/w), preferably less than 1% (w/w). The present invention also relates to special methods of preparing such powders and methods of administering them by inhalation.

### RELATED APPLICATIONS

Benefit of U.S. Provisional Application Ser. No. 60/503,115, filed on Sep. 15, 2003, is hereby claimed, and which application is incorporated herein in its entirety.

### SCOPE OF THE INVENTION

The invention relates to spray-dried amorphous powders containing protein/peptide which are stable on storage and processes for preparing them. The invention particularly relates to the preparation of mannitol-containing powders which contain protein/peptide by spray-drying and suitable after-drying.

### BACKGROUND

The progressive development of biotechnology has led to a major increase in the number of pharmaceutical preparations with active substances containing peptide or protein. Peptides/proteins are frequently subject to physical and chemical instability as soon as they spend any length of time in aqueous solutions (Cleland et al 1993, Crit. Rev. Ther. Drug Carrier Syst. 10(4), 307-377). The chemical instabilities include for example deamidation, hydrolysis, racemisation, oxidation, β-elimination and disulphide bridge exchange. Physical instabilities may be denaturing with subsequent aggregation or adsorption and direct aggregation followed by precipitation. These mechanisms which directly affect the structure of the peptide/protein usually lead to a reduction in its bioactivity and hence its therapeutic effect.

Peptides/proteins can be stabilised by drying processes. There are various known processes for preparing dry peptide/protein-containing pharmaceutical compositions. Freeze-drying methods are widespread, for example (Franks, et al., 1990, Cryo Lett., 11, 93-11; Pikal et al., 1990, Biopharm. 3(9), 26-30; Hora et al., 1992, Pharm. Res. 8(3), 285-291; Franks et al., 1992, Jap. J. Freezing Drying 38, 15-16; WO 02/101412). However, such processes are disadvantageous as many proteins are damaged on freezing or physiologically unacceptable excipients are needed to stabilise them. Alternatives to this are vacuum drying or spray drying processes (Franks et al., 1993, in van den Teel et al., (Eds.) Stability and Stabilisation of Enzymes, Elsevier Sci. Publ., 45-54; Roser et al., 1991, Biopharm. 4(9), 47-53; Carpenter et al., 1988; Cryobiol. 25, 459-470).

A process for chemically and physically stabilising peptides/proteins and hence for preparing powdered pharmaceutical preparations containing peptides/proteins which has become preferred in the meantime is spray-drying (cf. Maa et al., 1998, Pharmaceutical Research, 15(5), 768-775). Particularly in the field of pulmonary treatment spray drying is a suitable method of producing peptide/protein-containing powders for treating various diseases (U.S. Pat. No. 5,626,874; U.S. Pat. No. 5,972,388; Broadhead et al., 1994, J. Pharm Pharmacol., 46(6), 458-467). The administration of peptide/proteins by inhalation is an alternative to traditional methods of administration such as parenteral administration, even in systemic diseases, as pharmaceutical products taken by inhalation may develop not only a local but also a systemic activity (WO 99/07340). The prerequisite for this is that the average particle size is in the range from 1-10 μm, so that the particles can penetrate deep into the lungs and thus enter the bloodstream. DE-A-179 22 07 describes the preparation of spray dried particles which are sufficiently dispersible for medical application (inhalation). In the meantime a number of methods of producing correspondingly inhalable particles have become known in the prior art (WO 95/31479; WO 96/09814; WO 96/32096; WO 96/32149; WO 97/41833; WO 97/44013; WO 98/16205; WO 98/31346; WO 99/66903; WO 00/10541; WO 01/13893; Maa et al., 1998, supra; Vidgren et al., 1987, Int. J. Pharmaceutics, 35, 139-144; Niven et al., 1994, Pharmaceutical Research, 11(8), 1101-1109).

When producing inhalable powders by spray-drying, unfolding, aggregation and/or inactivation of the peptide/protein may occur (Broadhead et al., 1994, see above). Such effects occur both during the spraying process and during the subsequent storage of the powder. The reasons for this may be, for example, shear forces occurring, high spraying temperatures, surface effects, liquid/solid interactions and the like. Other problems may consist in the resulting dry powders having excessive mean particle sizes, poor flow qualities or poor dispersibility. For this reason peptide/proteins are sprayed with one or more excipients (or matrix forming agents) during spray-drying. The choice of the excipients depends among other things on their stabilising qualities. In addition, factors such as the pharmaceutical acceptance of the excipient and its effects on particle formation, particularly the mean particle size, the mean aerodynamic particle diameter (MMAD), the proportion of the fine particle fraction (FPF), the dispersibility and flow properties play a crucial role. Sugar and alcohols thereof such as, for example, trehalose, lactose, saccharose or mannitol and various polymers have proved suitable as excipients (Maa et al., 1997, Pharm. Development and Technology, 2(3), 213-223; Maa et al., 1998, supra; Dissertation Adler, 1998, Universität Erlangen; Costantino, et al., 1998, J. of Pharm. Sciences, 87(11), 1406-1411).

Costantino et al., describe how mannitol has a maximum stabilising effect on proteins in a proportion of from 20 to 80% (w mannitol/w protein). Although higher proportions of mannitol would lead one to expect improved flow properties and protein stabilisation, the actual tendency to protein stabilisation decreases as the proportion of mannitol increases as mannitol has a tendency to crystallise out. In formulations containing 10-30% (w/w) of mannitol the crystalline content increases during storage to more than 10% of the total protein content.

It is known from Maa et al., 1998 (see above) to produce mannitol-containing antibody formulations for inhalation which are prepared by spray-drying and after-drying. The residual moisture content for the formulation with 80/20% (w/w) anti-IgE antibody/mannitol was 2.4% (w/w) subsequent to after-drying (according to the Karl-Fischer method). The content of aggregates after long term storage over 15 weeks at 30° C. or 40° C. was up to 18% of the total protein content. The powder performance shown is thus not really suitable for the administration of pharmaceutical active substances.

The ability of polyols, particularly mannitol, to stabilise pharmaceutical active substances such as peptides or proteins sufficiently to form powders which perform well when inhaled appears to be greatly restricted by the high crystalline potential of the polyols,

One objective of the present invention was to make use of the stabilising effect of polyols, consisting of reducing crystallisation, for pharmaceutical applications, particularly for formulating spray-dried powders, containing peptides/proteins.

A further aim of the invention was to make use of the stabilising effect of polyols for formulating spray-dried powders which contain complex proteins such as, for example, antibodies as their pharmaceutical active substance.

A basic objective of the present invention was to provide spray-dried powders which are characterised by good long-term stability and inhalability. A balance between the two criteria is crucial.

Another aim of the present invention was to provide pharmaceutical preparations for administration by inhalation, either in the form of a dry powder or a propellant-containing metered dose aerosol or a propellant-free inhalant solution.

The objectives on which the invention is based are achieved by the embodiments described below and by the objects/methods recited in the claims.

### SUMMARY OF THE INVENTION

The present invention relates to spray-dried amorphous powders which contain a pharmaceutical active substance and a matrix-forming agent. The powders have a moisture content which is less than 1.2% (w/w), preferably less than 1% (w/w).

In a preferred embodiment, the powder consists predominantly of finely divided inhalable particles with a mass mean aerodynamic diameter (MMAD) of ≦10 μm, preferably 0.5-7.5 μm, more preferably 1-5 μm. The matrix forming agents may be sugars, polyols, polymers or a combination thereof. The sugars are preferably mono-, di-, oligo- or polysaccharides or a combination thereof.

In a particularly preferred embodiment the powders contain polyols as matrix forming agents, e.g. mannitol. The amount of mannitol in the powders is between 20 and 60% (w/w), according to another embodiment of the invention, preferably between 20-40% (w/w) of the dry mass of the powder. According to a preferred embodiment of the invention the polyol content, particularly the mannitol content, is more than 20% (w/w), preferably between 30-60% (w/w), more preferably between 30-50% (w/w), and even more preferably between, 30-40% (w/w).

The spray-dried powders according to the invention may additionally contain one or more surface-active substance(s) and/or a salt or salts. In addition, the spray-dried powders may contain other excipients such as for example amino acids, peptides, proteins or sugars. Preferably, the additional excipient is an amino acid, particularly isoleucine or a di- or tripeptide with at least one isoleucine group, preferably tri-isoleucine. According to a particular embodiment the present invention relates to spray-dried powders which contain, relative to their dry mass, a proportion of (a) approximately 20 to 60% (w/w) of matrix forming agent, preferably a polyol, such as mannitol, for example, (b) approximately 1 to 19.99% (w/w) of amino acids, preferably isoleucine and (c) approximately 0.01 to 79% (w/w) of a pharmaceutical active substance, preferably a peptide/protein, for example an antibody. According to another alternative embodiment the present invention relates to spray-dried powders which contain, in relation to their dry mass, (a) approximately 20 to 60% (w/w) of matrix forming agent, preferably a polyol, such as mannitol, for example, (b) approximately 1 to 19.99% (w/w) of a tripeptide that contains isoleucine, preferably tri-isoleucine and (c) approximately 0.01 to 79% (w/w) of a pharmaceutical active substance, preferably a peptide/protein, for example an antibody. The corresponding powders, particularly after the addition of isoleucine, or tripeptides that contain isoleucine, have very good flow properties and are characterised by a very high proportion of inhalable particles. In addition the corresponding powders are extremely stable during processing and storage.

The pharmaceutically active substance is preferably a biological macromolecule which may be a polypeptide or a protein. According to another preferred embodiment the powders according to the invention contain growth vectors, enzymes or antibodies. The invention relates in particular to inhalable spray-dried powders containing (a) a mannitol content of ≧20% (w/w), (b) antibodies as the pharmaceutically active substance, preferably in a concentration of 0.1-80% (w/w), and (c) a residual moisture content of ≦1.2% (w/w), preferably ≦1.0% (w/w).

According to another embodiment of the invention the content of aggregated active substance in the powders according to the invention is less than 3.5%, preferably less than 3% based on the total content of active substance.

The powders according to the invention are suitable for formulating pharmaceutical compositions, with the result that the present invention also includes corresponding pharmaceutical compositions, especially for inhalation, which contain one of the powders according to the invention described herein. In connection with this, pharmaceutical compositions which contain the powders according to the invention as propellant-containing metered dose aerosols, or as propellant-free inhalant solutions are particularly preferred.

The invention also provides a process consisting of spray drying and vacuum drying for producing protein preparations in the presence of at least one excipient. The temperature for this spray-drying process is below 135/70° C. (inflow/outflow temperature), preferably below 105/60° C. The resulting powder if after-dried by vacuum drying at a temperature of 25-60° C., preferably 30-60° C. The vacuum of the drying is adjusted to between 0.05 and 1 mbar, preferably between 0.05 and 0.5 mbar, most preferably between 0.05 and 0.2 mbar.

The present invention provides spray-dried amorphous powders with improved properties in terms of residual moisture content and the characteristics dependent thereon such as flow properties, dispersibility, shelf life and stability on processing. The present invention thus solves problems which have arisen during the development of the formulation hitherto, particularly when using spray-dried powders containing mannitol, as their residual moisture contents can have a negative effect on the physical and chemical stability of the powders. The activity of the peptides/proteins is retained during storage and during the process in the formulations.

### DESCRIPTION OF THE FIGURES

FIG. 1 describes the residual water content (=residual moisture) directly after spray drying at a room temperature of 22.5° C. and 50% r.h. (=relative humidity) and following after-drying in vacuum (24 h, 32° C., O. Imbar). It will be seen that the residual water content falls to below 1% in all the formulations. Without the after-drying the residual water content remains at 4 to 9.5%.

FIG. 2 shows aggregate formation after spray-drying and subsequent vacuum drying before storage, after 1, 4 and 15 weeks' storage at 2-8° C. sealed under nitrogen and welded into aluminium containers.

FIG. 3 shows aggregate formation after spray-drying with and without subsequent vacuum drying before storage and after 1, 4 and 15 weeks storage at 25° C./60% r.h. sealed under nitrogen and welded into aluminium containers. After-drying leads to substantial reduction in aggregate formation for powders with a mannitol content of 30% (w/w) or more.

FIG. 4 shows aggregate formation after spray-drying with and without subsequent vacuum drying, before storage, after 1, 4 and 15 weeks storage at 40° C./75% r.h. sealed under nitrogen and welded into aluminium containers. The after-drying leads to a substantial reduction in aggregate formation for powders with a mannitol content of 30% (w/w) or more.

FIG. 5 shows an inhaler for the administration of dry powdered preparations by inhalation.

### DETAILED DESCRIPTION OF THE INVENTION

The present invention is based on the fact that pharmaceutical active substances, particularly biological macromolecules, can be spray-dried with excipients and in this way inhalable powders which are stable on storage can be produced.

Definitions

The term “dry powder” refers to a composition of finely dispersed solid particles which are free-flowing. The term “dry” in this context means that the powders have a low residual moisture content of less than 10%, usually less than 5%, and preferably less than 3%.

The term “amorphous” means that the powdered formulation contains less than 10% crystalline fractions, preferably less than 7%, more preferably less than 5%, and most preferably less than 4, 3, 2, or 1%.

The word “inhalable” means that the powders are suitable for pulmonary administration. Inhalable powders can be dispersed and inhaled by means of an inhaler so that the particles enter the lungs and are able to develop a systemic activity optionally through the alveoli. Inhalable particles may have an average particle diameter, for example, of between 0.4-μm (MMD=mass medium diameter), usually between 0.5-4 μm, preferably between 1-3 μm and/or an average aerodynamic particle diameter (MMAD=mass median aerodynamic diameter) of between 0.5-10 μm, preferably between 0.5-7.5 μm, more preferably between 1-5 μm, even more preferably 1.5-4.5 μm and most preferably between 1.5-4 μm auf.

The term “dispersible” refers to the aerosol formation of the powder and indicates that the quantity delivered is more than 30%, preferably more than 40%, and preferably more than 50%. In a preferred embodiment the proportion delivered is more than 60%, preferably more than 70%, more preferably more than 75%, and better still more than 80%. The term “mean particle size” or “mean particle diameter” refers to the mean volumetric diameter and gives the volumetric particle size at which 50% of the particles of the powder have a smaller volumetric diameter. This thus corresponds to the mass median diameter (MMD). In cases of doubt the mean particle size can be determined using the method specified in this patent specification (cf. The chapter headed EXAMPLES, method).

The term mean aerodynamic particle diameter (=mass median aerodynamic diameter (MMAD)) indicates the aerodynamic particle size at which 50% of the particles of the powder have a smaller aerodynamic diameter. In cases of doubt the reference method for determining the MMAD is the method specified in this patent specification (cf. The chapter EXAMPLES, method).

The term “fine particle fraction” (FPF) describes the inhalable part of a powder consisting of particles with a particle size of ≦7.5 μm MMAD. In powder which is well dispersible the FPF is more than 20%, preferably more than 30%, more particularly more than 40%, and more preferably more than 45%, even more preferably more than 50%.

By a “pharmaceutical active substance” is meant a substance, medicine, composition or combination thereof which has a pharmacological, usually positive effect on an organism, an organ and/or a cell if the active substance is brought into contact with the organism, organ or cell. When introduced into a patient the effect may be local or systemic.

The term “biological macromolecule” refers to peptides, proteins, fats, fatty acids or nucleic acids.

The term “peptide” or “polypeptide” refers to polymers of amino acids consisting of two to a hundred amino acid groups. The term “peptide” or “polypeptide” is used as a pseudonym and includes both homo peptides and hetero peptides, i.e. polymers of amino acids consisting of one or a number of amino acid groups.

The term “protein” refers to polymers of amino acids with more than 100 amino acid groups. The term “protein” includes both single-chained polymers and multi-chained polymers such as antibodies, for example. This term also includes proteins which have been post-translationally modified by reactions such as glycosylation, phosphorylation, acetylation or protein processing. The structure of the polypeptide may be modified for example by substitution, deletion or insertion of amino acids, fusion with other proteins, while retaining its biological activity. The term “protein” thus also includes, for example, fusion proteins consisting of an immunoglobulin content, e.g. the Fc portion, and a growth factor, e.g. an interleukin.

The term “native conformation” means that after spray-drying and/or storage the peptide/protein retains its original secondary and tertiary structure or its original biological activity remains virtually unchanged.

The term “excipient” means an ingredient which stabilises the pharmaceutical active substance, particularly the biological macromolecules and/or improves the inhalability of the powders. Excipients for the purposes of the invention may be: sugars, polyols, polymers or a combination of these substances. The sugars are mono-, di-, oligo- or polysaccharides or combination thereof. Moreover, the term “excipient” includes amino acids, di-, tri-, oligo- or polypeptides as well as proteins, inorganic salts, organic salts or acids and surface active substances.

By matrix forming agents are meant excipients which essentially co-determine the shape and properties of the particles. Matrix forming agents may theoretically be sugars, polyols, polymers, amino acids, di- tri-, oligo-, polypeptides, proteins, or salts thereof.

The term “aggregates” refers to non-covalently bound dimers, trimers or oligomers or composites of a number of molecules. The proportion of aggregates in the powders according to the invention should be less than 5%, preferably less than 3.5%, and most preferably less than 2.5%.

The term “containing” also indicates the embodiment “consisting of” without mentioning this separately. “Containing a matrix forming agent and a pharmaceutical active substance” thus also refers to the embodiment “consisting of a matrix forming agent and a pharmaceutical active substance”.

Powder Composition

The present invention is based on the observation that advantageous amorphous powders can be prepared by spray-drying followed by after-drying, the spray-dried powders containing (a) at least one pharmaceutical active substance, preferably a biological macromolecule such as a peptide or protein, and (b) at least one matrix forming agent, preferably a polymer, sugar, sugar alcohol (=polyol) such as e.g. mannitol, or a combination thereof. Polymers, sugars and the alcohols thereof are particularly suitable as matrix forming agents in the preparation of the spray-dried powders according to the invention as they produce powders which disperse easily and have excellent stabilising properties.

Suitable polymers include for example polyvinylpyrrolidones, derivatised celluloses, such as hydroxymethyl, hydroxyethyl or hydroxypropylethyl cellulose, polymeric sugars such as Ficoll, starch such as hydroxyethyl or hydroxypropyl starch, dextrins such as cyclodextrins (2-hydroxypropyl-B-cyclodextrin, sulphobutylether-β-cyclodextrin), polyethylenes, glycols, chitosan, collagen, hyaluronic acid, polyacrylates, polyvinylalcohols, guar and/or pectins. The sugar is preferably a mono-, di-, oligo- or polysaccharide or a combination thereof. Examples of monosaccharides are fructose, maltose, galactose, glucose, d-mannose, sorbose and the like. Suitable disaccharides for the purposes of the invention include for example, lactose, sucrose, trehalose, cellobiose, and the like. Polysaccharides which may be used include in particular raffinose, melecitose, dextrin, starch and the like. Sugar alcohols include in addition to mannitol (which is preferred), xylitol, maltitol, galactitol, arabinitol, adonitol, lactitol, sorbitol (glucitol), pyranosylsorbitol, inositol, myoinositol and the like as matrix forming agents.

Surprisingly, it has been found that corresponding spray-dried powders have particularly advantageous properties if their residual water content (=residual moisture) is less than 1.2% (w/w), preferably less than 1.0% (w/w). High residual moisture contents lower the glass transition temperature (Tg) of an amorphous matrix and thus favour the formation of partially crystalline or fully crystalline structures. If the storage temperature exceeds the glass transition temperature the amorphous matrix changes into a rubber-like state with increased molecule mobility and reactivity.

Accordingly, the present invention also relates to spray-dried amorphous powders containing (a) at least one pharmaceutical active substance, preferably a biological macromolecule such as e.g. peptides or proteins, and (b) at least one matrix forming agent, preferably a polymer, a sugar, sugar alcohol (=polyol) such as e.g. mannitol, or a combination thereof, characterised in that the residual water content of the powder is ≦1.2% (w/w), preferably ≦1.0% (w/w).

However, high spraying temperatures give rise to problems in terms of the denaturing and aggregation of pharmaceutical active substances, particularly peptides/proteins. There is thus a certain limit to the reduction of the residual water content (residual humidity) during the spraying process. The more complex the structure of a peptide or protein the more unstable it is in the presence of the effects of heat. Thus, small peptides or single-chained proteins, for example, are substantially more heat-resistant than antibodies. Consequently it is difficult to find the correct balance between the amount of excipient and the spray-drying temperature used. Excipients have a stabilising affect on the active substance on the one hand and permit higher spray-drying temperatures. However, on the other hand, because of the hydroscopic properties they may favour the absorption of moisture which in turn leads to crystallisation of the active substance. For this reason most spray dried powders, particularly if they contain complex peptides/protein as pharmaceutical active substance, have residual moisture contents of roughly 2% (w/w) or even more (U.S. Pat. No. 6,165,463; U.S. Pat. No. 6,019,968; Maa et al., 1998, supra). These residual moisture contents are disadvantageous in terms of dispersibility and long term stability.

It was known that sugars in particular and their alcohols are suitable for stabilising proteins. Because of their hygroscopic properties sugars and their alcohols have hitherto only proved advantageous in concentrations of up to 20% (w/w) (cf. Maa et al., 1998, see above). Admittedly, powder preparations with a higher content of sugar or sugar alcohol have been described but because of their relatively high residual moisture contents they have limited dispersibility and long term stability. The present invention for the first time provides powders which exploit the advantageous properties of sugar and/or sugar alcohols in the stabilising of active substances in concentrations of more than 20% (w/w), preferably 30-60% (w/w), more preferably 30-50% (w/w), and in particular 30-40% (w/w). Accordingly, the present invention relates to spray-dried powders containing (a) at least one pharmaceutical active substance and (b) at least one matrix forming agent, characterised in that the matrix forming agent consists of sugar, sugar alcohols (polyols) and/or a combination of these. Those powders which contain polyols such as mannitol as matrix forming agent are particularly preferred.

Also suitable are sugars and/or sugar alcohols which have a Tg value of more than 40° C., preferably more than 45° C., even more preferably more than 50° C., for example more than 55, 60, 65, 70, 75, 80, 85, 90° C., etc., in the concentration used. Consequently, in another embodiment, the present invention also relates to amorphous spray-dried powders containing (a) at least one pharmaceutical active substance, preferably a biological macromolecule such as peptides or proteins, for example and (b) at least one matrix forming agent, preferably a sugar or sugar alcohol (=polyol) such as mannitol, characterised in that the Tg of the powder is above 40° C., preferably above 45° C., more preferably above 50° C., even more preferably above 55° C., for example above 60, 65, 70, 75, 80, 85, 90° C. A corresponding powder which contains mannitol as matrix forming agent is particularly preferred. The Tg of a powder can be determined experimentally by DCS (differential scanning calorimetry) (Breen et al., 2001, Pharm. Res., 18(9), 1345-1353). The increase in the heat capacity is shown as a function of the temperature. As globular proteins show only slight changes in the heat capacity at the Tg, the Tg values are measured by modulating DSC.

Generally, the proportion of the corresponding matrix forming agent in the powders according to the invention is between 20-60% (w/w), more preferably between 20-40% (w/w) of the dry mass of the powder. According to another preferred embodiment of the invention the proportion of the corresponding matrix forming agent, e.g. the polyol or mannitol content is more than 20% (w/w) of the dry mass of the powder, preferably between 30-60% (w/w), more preferably between 30-50% (w/w), even more preferably between 30-40% (w/w) and more preferably still between 35-40% (w/w). As will be apparent from the examples that follow, an increase in the matrix content to more than 20% (w/w), preferably to 30-60% (w/w) with a high dispersibility does not lead to any substantial increase in aggregates. According to these embodiments, the proportion of the matrix in question can therefore be roughly 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59 or 60% (w/w) of the dry mass of the powder. The corresponding embodiments apply particularly to powders in which polyols and particularly mannitol is used as the matrix forming agent.

According to a preferred embodiment the powder according to the invention contains only one of the sugars, polyols or polymers listed above as matrix forming agent in one of the concentrations specified, while other sugars, polyols or polymers may be added to the powders according to the invention in smaller amounts of up to 20% (w/w), preferably up to 10% (w/w), most preferably up to 5% (w/w). Preferably, the matrix forming agent is mannitol in one of the concentrations specified above.

According to another embodiment according to the invention the total proportion of sugar, polyol, and/or polymer in the powder is at most 99% (w/w), preferably at most 95% (w/w), more preferably at most 90% (w/w), even more preferably at most 80% (w/w). In a preferred embodiment the proportion of sugar, polyol and/or polymer in the powder is not more than 70% (w/w), preferably not more than 60% (w/w), preferably not more than 50% (w/w), more preferably between 40 and 30% (w/w).

The combination of low residual moisture content—as described above—and the use of corresponding matrix forming agents—as specified above—surprisingly led to highly dispersible powders which are stable on storage. The proportion of aggregates after spray drying and after drying in the powders according to the invention with a matrix content of between 20-60% was less then 2%. Even after 15 weeks storage at 30 or 40° C. and at a relative humidity of 60% or 75% the proportion of aggregates did not rise above 3.5%. Powders with higher residual moisture contents had a comparatively high proportion of aggregates (between 7 and 13% after only 4 weeks storage at 40° C. and 75% r.h.). These values also coincide with the observations of Maa et al 1998 (see above).

The powders according to the invention may additionally contain salts, especially pharmaceutically acceptable salts. These may be for example inorganic salts such as chlorides, sulphates, phosphates, diphosphates, hydrobromides and/or nitrate salts. Moreover the powders according to the invention may also contain organic salts, such as e.g. malates, maleates, fumarates, tartrates, succinates, ethylsuccinates, citrates, acetates, lactates, methanesulphonates, benzoates, ascorbates, paratoluenesulphonates, palmoates, salicylates, stearates, estolates, gluceptates or labionate salts. At the same time corresponding salts may contain pharmaceutically acceptable cations, such as for example sodium, potassium, calcium, aluminium, lithium or ammonium. It is particularly preferred to use corresponding cations in conjunction with the stabilisation of proteins as well as a powder containing mannitol in combination with citrate. Citrate concentrations of between 0.1 and 150 mM (based on the solution to be sprayed) are particularly preferred.

According to another embodiment according to the invention the powders may additionally contain surfactants such as Tween 20, 40, 60, 80, Brij 35, Pluronic F 88 and Pluronic F 127. These are preferably used in a concentration of 0.01-0.1% (w/w). Particularly preferred is a spray-dried powder that contains as matrix-forming agent a sugar or a polyol, particularly mannitol and additionally Tween 20, preferably in a concentration of 0.01-0.1% (w/w), as surface-active substance. Powders containing roughly 29.95% (w/w) mannitol, 69.95% (w/w) of an antibody and 0.1% (w/w) Tween 20 had an aggregate content of less than 1% after long-term storage and proved to be particularly advantageous for the purposes of the invention.

The powders according to the invention may additionally contain other excipients, such as for example amino acid, peptides, non-biological or biological polymers, and/or one or more of the sugars listed above. Other excipients known in the art are for example lipids, fatty acids, fatty acid esters, steroids (e.g. cholesterol) or chelating agents (e.g. EDTA) as well as various cations (see above). Excipients with a high glass transition temperature, for example above 40° C., preferably above 45° C., or above 55° C., are particularly preferred. A list of suitable excipients can be found for example in Kippe (Eds.), “Handbook of Pharmaceutical Excipient” 3rd Ed., 2000.

Suitable protein-containing excipients include for example albumin (human or recombinant in origin), gelatine, casein, haemoglobin and the like. Suitable amino acids include for example alanine, glycine, arginine, histidine, glutamate, asparagine, cysteine, leucine, lysine, isoleucine, valine, tryptophan, methionine, phenylalanine, tyrosine, L-aspartyl-L-phenylalanine-methylester (=aspartame), trimethylammonioacetate (=betaine) and the like. Preferably, amino acids are used which act as buffers (e.g. glycine or histidine) and/or as dispersing agents. These latter groups include in particular predominantly hydrophobic amino acids, such as e.g. leucine, valine, isoleucine, tryptophan, alanine, methionine, phenylalanine, tyrosine, histidine or proline.

Within the scope of the present invention the use of isoleucine as an additional excipient has proved particularly advantageous. It isparticularly advantageous to use isoleucine in a concentration of 1 to 19.99% (w/w), preferably from 5 to 19.99% (w/w), still more preferably from 10 to 19.99% (w/w). The proportion of [sic] may, however, also be increased to levels of up to 30% (w/w), provided that the proportion of the matrix forming agent or the proportion of pharmaceutical active substance is reduced accordingly, so that the solid content of the powder is a maximum of 100% (w/w).

It is also advantageous to use di-, tri-, oligo- or polypeptides as excipients which contain one or more of these predominantly hydrophobic amino acid groups. Suitable examples of di- or tri-peptides can be found inter alia in WO 01/32144, the contents of which are hereby incorporated by reference. These may be for example one or more of the following tripeptides: Leu-Leu-Gly, Leu-Leu-Ala, Leu-Leu-Val, Leu-Leu-Leu, Leu-Leu-Met, Leu-Leu-Pro, Leu-Leu-Phe, Leu-Leu-Trp, Leu-Leu-Ser, Leu-Leu-Thr, Leu-Leu-Cys, Leu-Leu-Tyr, Leu-Leu-Asp, Leu-Leu-Glu, Leu-Leu-Lys, Leu-Leu-Arg, Leu-Leu-His, Leu-Gly-Leu, Leu-Ala-Leu, Leu-Val-Leu, Leu-Met-Leu, Leu-Pro-Leu, Leu-Phe-Leu, Leu-Trp-Leu, Leu-Ser-Leu, Leu-Thr-Leu, Leu-Cys-Leu, Leu-Try-Leu, Leu-Asp-Leu, Leu-Glu-Leu, Leu-Lys-Leu, Leu-Arg-Leu und Leu-His-Leu. It has proved particularly advantageous to use tripeptides of the general formulae: Ile-X-X; X-Ile-X; X-X-Ile, wherein X may be one of the following amino acids: alanine, glycine, arginine, histidine, glutamic acid, glutamine, asparagine, aspartic acid, cysteine, leucine, lysine, isoleucine (Ile), valine, tryptophan, methionine, phenylalanine, proline, serine, threonine, tyrosine, L-aspartyl-L-phenylalanine-methylester (=aspartame), trimethylammonio-acetate and Ile denotes isoleucine. Particularly preferred are corresponding tripeptides of the formula (Ile)₂-X, for example Ile—Ile-X, Ile-X-Ile, or X-Ile-Ile, wherein X in turn may be one of the above-mentioned amino acids. These include for example the tripeptides: Ile-Ile-Gly, Ile-Ile-Ala, Ile-Ile-Val, Ile-Ile-Ile, Ile-Ile-Met, Ile-Ile-Pro, Ile-Ile-Phe, Ile-Ile-Trp, Ile-Ile-Ser, Ile-Ile-Thr, Ile-Ile-Cys, Ile-Ile-Tyr, Ile-Ile-Asp, Ile-Ile-Glu, Ile-Ile-Lys, Ile-Ile-Arg, Ile-Ile-His, Ile-Gly-Ile, Ile-Ala-Ile, Ile-Val-Ile, Ile-Met-Ile, Ile-Pro-Ile, Ile-Phe-Ile, Ile-Trp-Ile, Ile-Ser-Ile, Ile-Thr-Ile, Ile-Cys-Ile, Ile-Try-Ile, Ile-Asp-Ile, Ile-Glu-Ile, Ile-Lys-Ile, Ile-Arg-Ile, Ile-His-Ile. The use of Ile-Ile-Ile is particularly advantageous.

According to another embodiment the present invention therefore relates to spray-dried powders which contain in relation to their dry mass a) a matrix forming agent, preferably a polyol such as for example mannitol in a concentration of 20-60% (w/w), particularly preferably in a concentration of 25-50% (w/w), still more preferably in a concentration of 30-40% (w/w) b) between 1 and 19.99% (w/w) amino acids and c) between 0.01 and 79% (w/w), preferably between 0.01 and 69% (w/w) of a pharmaceutical active substance, preferably a peptide/protein, for example an antibody, the sum of the parts by weight making up at most 100% (w/w). Consequently in another embodiment the present invention also relates to powders which contain or consist of for example 20% (w/w) of matrix forming agent, preferably polyol, such as for example mannitol/10% (w/w) amino acid/70% (w/w) pharmaceutical active substance, preferably a peptide/protein, for example an antibody, (20/10/70); or (21/10/69) for example (22/10/68); (23/10/67); (24/10/66); (25/10/65); (26/10/64); (27/10/63); (28/10/62); (29/10/61); (30/10/60); (31/10/59); (32/10/58); (33/10/57); (34/10/56); (35/10/55); (36/10/54); (37/10/53); (38/10/52); (39/10/51) or (40/10/50). If the amino acid content varies between 1 and 20% (w/w), with a constant amount of for example 20% (w/w) of mannitol, for example if it is 1, 2, 3 . . . 8, 9, 10, 11, 12, 13 . . . 18, 19, 19.1, 19.2, 19.3 . . . 19.8, 19.9, 19.91, 19.92, 19.93, . . . 19.97, 19.98, 19.99% (w/w), the proportion of pharmaceutical active substance, preferably a peptide/protein, for example an antibody, is reduced, from a starting point of 79% (w/w) to 78, 77, . . . 72, 71, 70, 69, 68, 67, . . . 63, 62, 61, 60.9, 60.8, 60.7 . . . 60.3, 60.2, 60.1, 60.09, 60.08, . . . 60.03, 60.02, 60.01% (w/w). If the proportion of matrix forming agent increases from a starting point of 20 to 39% (w/w), for example to 21, 22, 23, 24, 25, 26, 27, 28, 29, 30, 31, 32, 33, 34, 35, 36, 37, 38, 38.1, 38.2, 38.3 . . . 38.8, 38.9, 38.91, 38.92 . . . 38.97, 38.98, 38.99, 39% (w/w), while the proportion of pharmaceutical active substance remains constant at 60% (w/w) for example, the amino acid content is reduced from a starting point of 20 to 1% (w/w), for example to 19, 18, 17, . . . 13, 12, 11, 10, 9, 8, 7, . . . 3, 2, 1.9, 1.8, 1.7 . . . 1.3, 1.2, 1.1, 1.09, 1.08, . . . 1.03, 1.02, 1.01, 1% (w/w), so that the sum of the parts by weight of the individual powdered ingredients in relation to the dry mass of the powder is a maximum of 100% (w/w). It has proved particularly advantageous to use mannitol as the matrix forming agent in a range from 21 to 50% (w/w), preferably from 25 to 50% (w/w), more preferably from 30 to 40% (w/w). By adding other excipients or salts the proportion of mannitol, amino acids and/or pharmaceutical active substance can be adjusted/reduced accordingly, so that the parts by weight of the individual ingredients add up to not more than 100% (w/w).

If the amino acid added is isoleucine, according to another embodiment the powders according to the invention contain a) a matrix forming agent, preferably a polyol, for example mannitol, in a concentration of 20-60% (w/w), particularly preferably in a concentration of 25-50% (w/w), still more preferably in a concentration of 30-40% (w/w) b) between 1 and 19.99% (w/w) of isoleucine and c) between 0.01 and 79% (w/w), preferably between 0.01 and 69% (w/w) of a pharmaceutical active substance, preferably a peptide/protein, for example an antibody, the parts by weight adding up to a maximum of 100% (w/w). Consequently, in another embodiment, the present invention also relates to powders which contain or consist of, for example, 20% (w/w) of matrix forming agent, preferably a polyol such as for example mannitol/10% (w/w) isoleucine/70% (w/w) pharmaceutical active substance (20/10/70); or for example (21/10/69); (22/10/68); (23/10/67); (24/10/66); (25/10/65); (26/10/64); (27/10/63); (28/10/62); (29/10/61); (30/10/60); (31/10/59); (32/10/58); (33/10/57); (34/10/56); (35/10/55); (36/10/54); (37/10/53); (38/10/52); (39/10/51) or (40/10/50). If the isoleucine content varies between 1 and 20% (w/w), for example if it is 1, 2, 3 . . . 8, 9, 10, 11, 12, 13 . . . 18, 19, 19.1, 19.2, 19.3 . . . 19.8, 19.9, 19.91, 19.92, 19.93, . . . 19.97, 19.98, 19.99% (w/w), while the proportion of matrix forming agent such as mannitol, for example, remains constant at least 20% (w/w), the proportion of pharmaceutical active substance, preferably a peptide/protein, decreases from a starting point of 79% (w/w) to 78, 77, . . . 72, 71, 70, 69, 68, 67, . . . 63, 62, 61, 60.9, 60.8, 60.7 . . . 60.3, 60.2, 60.1, 60.09, 60.08, . . . 60.03, 60.02, 60.01% (w/w). If the proportion of matrix forming agent such as mannitol, for example, is increased from a starting point of 20 to 39% (w/w), for example to 21, 22, 23, 24, 25, 26, 27, 28, 29, 30, 31, 32, 33, 34, 35, 36, 37, 38, 38.1, 38.2, 38.3 . . . 38.8, 38.9, 38.91, 38.92 . . . 38.97, 38.98, 38.99, 39% (w/w), while the proportion of pharmaceutical active substance, preferably a protein/peptide, remains constant at 60% (w/w), the isoleucine content is reduced from a starting point of 20 to 1% (w/w), for example to 19, 18, 17, . . . 13, 12, 11, 10, 9, 8, 7, . . . 3, 2, 1.9, 1.8, 1.7 . . . 1.3, 1.2, 1.1 1.09, 1.08, . . . 1.03, 1.02, 1.01, 1% (w/w), so that the sum of the parts by weight of the individual powder ingredients in relation to the dry mass of the powder is a maximum of 100% (w/w). It has proved particularly advantageous to use mannitol as the matrix forming agent in a range from 21 to 50% (w/w), preferably from 25 to 50% (w/w), more preferably from 30 to 40% (w/w). By adding other excipients or salts, the amount of mannitol, isoleucine and/or pharmaceutical active substance can be adjusted/reduced accordingly, so that the parts by weight of the individual ingredients add up to not more than 100% (w/w).

Furthermore, according to another embodiment, the present invention also relates to powders a) having a matrix forming agent, preferably a polyol, for example mannitol, in a concentration of 20-60% (w/w), particularly preferably in a concentration of 25-50% (w/w), still more preferably in a concentration of 30-40% (w/w), b) between 1 and 19.99% (w/w) of di- or tri-peptide, preferably with one or more isoleucine groups and c) between 0.01 and 79% (w/w), preferably between 0.01 and 69% (w/w) of a pharmaceutical active substance, preferably a peptide/protein, for example an antibody, the sum of the parts by weight being at most 100% (w/w). Consequently, according to another embodiment, the present invention also relates to powders which contain or consist of, for example, 20% (w/w) of matrix forming agent, preferably polyol, such as for example mannitol/10% (w/w) of di- or tri-peptide, preferably with one or more isoleucine groups/70% (w/w) of pharmaceutical active substance=(20/10/70); or for example (21/1069); (22/10/68); (23/10/67); (24/10/66); (25/10/65); (26/10/64); (27/10/63); (28/10/62); (29/10/61); (30/10/60); (31/10/59); (32/10/58); (33/10/57); (34/10/56); (35/10/55); (36/10/54); (37/10/53); (38/10/52); (39/10/51) or (40/10/50). If the proportion of the said di- or tripeptides varies between 1 and 20% (w/w), and is for example 1, 2, 3 . . . 8, 9, 10, 11, 12, 13 . . . 18, 19, 19.1, 19.2, 19.3 . . . 19.8, 19.9, 19.91, 19.92, 19.93, . . . 19.97, 19.98, 19.99% (w/w), while the proportion of matrix forming agent, preferably mannitol, remains constant at least 20% (w/w), the proportion of pharmaceutical active substance, preferably peptide/protein, for example an antibody decreases from a starting point of 79% (w/w) to 78, 77, . . . 72, 71, 70, 69, 68, 67, . . . 63, 62, 61, 60.9, 60.8, 60.7 . . . 60.3, 60.2, 60.1, 60.09, 60.08, . . . 60.03, 60.02, 60.01% (w/w). If the proportion of matrix forming agent, preferably a polyol such as mannitol, for example, increases from a starting point of 20 to 39% (w/w), for example to 21, 22, 23, 24, 25, 26, 27, 28, 29, 30, 31, 32, 33, 34, 35, 36, 37, 38, 38.1, 38.2, 38.3 . . . 38.8, 38.9, 38.91, 38.92 . . . 38.97, 38.98, 38.99, 39% (w/w), while the proportion of pharmaceutical active substance remains constant at 60% (w/w), the proportion of di- or tripeptide, preferably with one or more isoleucine groups, decreases from a starting point of 20 to 1% (w/w), for example to 19, 18, 17, . . . 13, 12, 11, 10, 9, 8, 7, . . . 3, 2, 1.9, 1.8, 1.7 . . . 1.3, 1.2, 1.1, 1.09, 1.08, . . . 1.03, 1.02, 1.01, 1% (w/w), so that the sum of the parts by weight of the individual powder ingredients in relation to the dry mass of the powder is at most 100% (w/w). It has proved particularly advantageous to use mannitol as the matrix forming agent in a range from 21 to 50% (w/w), preferably from 25 to 50% (w/w), more preferably from 30 to 40% (w/w). By adding other excipients or salts the amount of mannitol, di- or tri-peptide, preferably with one or more isoleucine groups and/or pharmaceutical active substance can be adjusted/reduced accordingly, so that the parts by weight of the individual ingredients add up to at most 100% (w/w).

If the tripeptide added is a tri-isoleucine, according to another embodiment the invention relates to powders having a) a matrix forming agent, preferably a polyol, for example mannitol, in a concentration of 20-60% (w/w), particularly preferably in a concentration of 25-50% (w/w), still more preferably in a concentration of 30-40% (w/w), b) between 1 and 19.99% (w/w) of tri-isoleucine and c) between 0.01 and 79% (w/w), preferably between 0.01 and 69% (w/w) of a pharmaceutical active substance, preferably a peptide/protein such as for example an antibody, the sum of the parts by weight being not more than 100% (w/w). Consequently, in another embodiment, the present invention also relates to powders which contain or consist of for example 20% (w/w) of matrix forming agent, preferably polyol, such as for example mannitol/10% (w/w) of tri-isoleucine/70% (w/w) pharmaceutical active substance (21/10/69); or for example (22/10/68); (23/10/67); (24/10/66); (25/10/65); (26/10/64); (27/10/63); (28/10/62); (29/10/61); (30/10/60); (31/10/59); (32/10/58); (33/10/57); (34/10/56); (35/10/55); (36/10/54); (37/10/53); (38/10/52); (39/10/51) or (40/10/50). If the tri-isoleucine content varies between 1 and 20% (w/w), for example is 1, 2, 3 . . . 8, 9, 10, 11, 12, 13 . . . 18, 19, 19.1, 19.2, 19.3 . . . 19.8, 19.9, 19.91, 19.92, 19.93, . . . 19.97, 19.98, 19.99% (w/w), while the proportion of matrix forming agent such as mannitol, for example, remains constant at least 20% (w/w), the proportion of pharmaceutical active substance preferably a peptide/protein, for example an antibody, decreases from a starting point of 79% (w/w) to 78, 77, . . . 72, 71, 70, 69, 68, 67, . . . 63, 62, 61, 60.9, 60.8, 60.7 . . . 60.3, 60.2, 60.1, 60.09, 60.08, . . . 60.03, 60.02, 60.01% (w/w). If the proportion of matrix forming agent, preferably a polyol such as mannitol, for example, increases from a starting point of 20 to 39% (w/w), for example to 21, 22, 23, 24, 25, 26, 27, 28, 29, 30, 31, 32, 33, 34, 35, 36, 37, 38, 38.1, 38.2, 38.3 . . . 38.8, 38.9, 38.91, 38.92 . . . 38.97, 38.98, 38.99, 39% (w/w), while the proportion of pharmaceutical active substance, preferably a protein/peptide, remains constant at 60% (w/w), the tri-isoleucine content is reduced from a starting point of 20 to 1% (w/w), for example to 19, 18, 17, . . . 13, 12, 11, 10, 9, 8, 7, . . . 3, 2, 1.9, 1.8, 1.7 . . . 1.3, 1.2, 1.1, 1.09, 1.08, . . . 1.03, 1.02, 1.01, 1% (w/w), so that the sum of the parts by weight of the individual powder ingredients in relation to the dry mass of the powder is at most 100% (w/w). The use of mannitol as matrix forming agent in a range from 21 to 50% (w/w), preferably from 25 to 50% (w/w), more preferably from 30 to 40% (w/w), is particularly advantageous. By adding other excipients or salts the amount of mannitol, tri-isoleucine and/or pharmaceutical active substance can be adjusted/reduced accordingly, so that the parts by weight of the individual ingredients add up to at most 100% (w/w).

According to another embodiment a combination of (a) mannitol, preferably in a concentration of 20-60% (w/w), particularly preferably in a concentration of 30-50% (w/w), even more preferably in a concentration of 30-40% (w/w) and (b) histidine or glycine and (c) a pharmaceutical active substance, preferably a protein or peptide, particularly preferably an antibody, has proved suitable. It has proved particularly advantageous to spray-dry solutions which contained in addition to the pharmaceutical active substance and mannitol 1.6 mM glycine and 25 mM histidine.

Suitable polymers comprise for example the polyvinylpyrrolidones which have already been mentioned above as matrix-forming agents, derivatised celluloses, such as e.g. hydroxymethyl-, hydroxyethyl-, or hydroxypropyl-ethylcellulose, polymeric sugars such as e.g. ficoll, starch such as e.g. hydroxyethyl or hydroxypropyl starch, dextrins such as e.g. cyclodextrins (2-hydroxypropyl-B-cyclodextrin, sulphobutylether-13-cyclodextrin), polyethylenes, glycols and/or pectins.

The pharmaceutical active substance is preferably a biological macromolecule. In accordance with the definition provided above this is intended to include for example peptides, proteins, fats, fatty acids or nucleic acids.

Biopharmaceutically important proteins/polypeptides include e.g. antibodies, enzymes, growth factors, e.g. cytokines, lymphokines, adhesion molecules, receptors and the derivatives or fragments thereof, but are not restricted thereto. Generally, all polypeptides which act as agonists or antagonists and/or have therapeutic or diagnostic applications are of value.

Suitable peptides or proteins for the purposes of the invention include for example insulin, insulin-like growth factor, human growth hormone (hGH) and other growth factors, tissue plasminogen activator (tPA), erythropoietin (EPO), cytokines, e.g. interleukines (IL) such as IL-1, IL-2, IL-3, IL-4, IL-5, IL-6, IL-7, IL-8, IL-9, IL-10, IL-11, IL-12, IL-13, IL-14, IL-15, IL-16, IL-17, IL-18, interferon (IFN)-alpha, -beta, -gamma, -omega or -tau, tumour necrosis factor (TNF) such as TNF-alpha, -beta or -gamma, TRAIL, G-CSF, GM-CSF, M-CSF, MCP-1 and VEGF. Other examples are monoclonal, polyclonal, multispecific and single chain antibodies and fragments thereof such as for example Fab, Fab′, F(ab′)₂, Fc and Fc′ fragments, light (L) and heavy (H) immunoglobulin chains and the constant, variable or hypervariable regions thereof as well as Fv and Fd fragments (Chamov et al., 1999, Antibody Fusion proteins, Wiley-Liss Inc.). The antibodies may be of human or non-human origin. These include for example the classes known in man: IgA, IgD, IgE, IgG and IgM, with their various subclasses, for example IgA1, IgA2 and IgG1, IgG2, IgG3 and IgG4. Humanised and chimeric antibodies are also possible. Of particular therapeutic importance and hence a subject of the present invention are powder formulations which [contain] antibodies against for example various surface antigens such as CD4, CD20 or CD44, various cytokines, for example IL2, IL4 or IL5. Other Examples are antibodies against specific classes of immunoglobulin (e.g. anti-IgE antibodies) or against viral proteins (e.g. anti-RSV, anti-CMV antibodies, etc.).

Fab fragments (fragment antigen binding=Fab) consist of the variable regions of both chains which are held together by the adjacent constant regions. Other antibody fragments are F(ab′)₂ fragments which can be produced by proteolytic digestion with pepsin. By gene cloning it is also possible to prepare shortened antibody fragments consisting of only the variable region of the heavy (VH) and light chain (VL). These are known as Fv fragments (fragment variable=fragment of the variable part). Such antibody fragments are also referred to as single chain Fv fragments (scFv). Examples of scFv antibodies are known and described, cf. for example Huston et al., 1988, Proc. Natl. Acad. Sci. USA, 16, 5879ff.

In past years various strategies have been developed for producing multimeric scFv derivatives, such as e.g. dia-, tri- and pentabodies. The term diabody is used in the art to denote a bivalent homodimeric scFv derivative. Shortening the peptide linker in the scFv molecule to 5 to 10 amino acids results in the formation of homodimers by superimposing VH/VL chains. The diabodies may additionally be stabilised by inserted disulphite bridges. Examples of diabodies can be found in the literature, e.g. in Perisic et al., 1994 (Structure, 2, 1217ff). The term minibody is used in the art to denote a bivalent homodimeric scFv derivative. It consists of a fusion protein which contains the CH3 region of an immunoglobulin, preferably IgG, most preferably IgG1, as dimerisation region. This connects the scFv fragments by means of a hinge region, also of IgG, and a linker region. Examples of such minibodies are described by Hu et al., 1996, Cancer Res., 56, 3055ff.

The term triabody is used in the art to denote a trivalent homotrimeric scFv derivative (Kortt et al., 1997, Protein Engineering, 10, 423ff). The direct fusion of VH-VL without the use of a linker sequence leads to the formation of trimers.

The fragments known in the art as mini antibodies which have a bi-, tri- or tetravalent structure are also derivatives of scFv fragments. The multimerisation is achieved by means of di-, tri- or tetrameric coiled coil structures (Pack, P. et al., 1993, Biotechnology, 11, 1271ff; Lovejoy, B. et al., 1993, Science, 259, 1288ff, Pack, P. et al., 1995, J. Mol. Biol., 246, 28ff).

The corresponding active substances may make up between 0.01 to 80% (w/w) of the total weight of the powder. Particularly advantageous are powders which contain as active substance a peptide or protein or a combination of peptide/peptide, peptide/protein or protein/protein. If the powders according to the invention contain growth factors, for example cytokines, the content is normally between 0.1 to 10% (w/w), preferably between 0.2 to 5% (w/w) of the total weight of the powder. Accordingly, powders wherein the content of cytokines is 0.2, 0.3, 0.4 . . . 0.8, 0.9 etc.; 1, 2, 3, . . . etc; 4.1, 4.2, 4.3, . . . 4.8, 4.9 etc.; 4.91, 4.92, 4.93, . . . 4.98, 4.99% (w/w) are preferred. If the pharmaceutical active substance is an antibody or a derivative thereof (preferred embodiment), the active substance content is between 0.1 and 80% (w/w), preferably between 1 and 80% (w/w), particularly preferably between 10 and 80% (w/w), more preferably between 30 and 80% (w/w), even more preferably between 60 and 80% (w/w), for example 0.1, 0.2, 0.3, 0.33, . . . 0.66, 0.7, 0.8, 0.9 etc.; 1, 2, 3, . . . 8, 9, 10 etc.; 11, 12, 13, . . . 18, 19, 20 etc.; 21, 22, 23, . . . 28, 29, 30 etc.; 31, 32, 33, . . . 38, 39, 40 etc.; 41, 42, 43, . . . 48, 49, 50 etc. 51, 52, 53, . . . 58, 59, 60 etc.; 61, 62, 63, . . . 68, 69, 70 etc.; 71, 72, 73, . . . 78, 79, etc; 79.1, 79.2, 79.3, . . . 79.8, 79.9 etc.; 79.91, 79.92, 79.93, . . . 79.98, 79.99, 80% (w/w) of the total weight of the powder.

Particularly advantageous powders according to the invention are those with a ratio of matrix-forming agent to peptide/protein of 21/79, 22/78, 23/77, 24/76, 25/75, 26/74, 27/73, 28/72, 29/71, 30/70, 31/69, 32/68, 33/67, 34/66, 35/65, 36/64, 37/63, 38/62, 39/61, 40/60, 41/59, 42/58, 43/57, 44/56, 45/55, 46/54, 47/53, 48/52, 49/51, 50/50, 51/49, 52/48, 53/47, 54/46, 55/45, 56/44, 57/43, 58/42, 59/41, 60/40, the data referring to the absolute amounts in the powder. If the corresponding powder contains one or more additional excipients, for example the amino acids and di- or tripeptides described above, either the amount of matrix-forming agent, the amount of pharmaceutical active substance, or both amounts may be reduced accordingly, while the amount of matrix-forming agent has one of the values between 21 and 60% (w/w), preferably between 25 and 50% (w/w), more preferably between 30 and 40% (w/w).

The matrix-forming agent may be, in particular, one of those mentioned in this application. It is particularly advantageous to use powders which contain (a) as active substance one of the above-mentioned peptides/proteins, preferably an antibody or a derivative thereof, and (b) a sugar, polyol and/or polymer, preferably a polyol, particularly preferably mannitol, in the proportions specified above, the amounts in each case referring to the absolute amount in the powder.

Powders which have proved particularly advantageous and therefore in accordance with the invention are those which (a) contain as active substance an antibody and (b) contain as matrix-forming agent a polyol, particularly mannitol in a concentration of more than 20% (w/w), preferably 25-60% (w/w), particularly preferably 25-50% (w/w), even more preferably 30-40% (w/w), and (c) the residual water content of said powder is below 1.2% (w/w), preferably below 1.0% (w/w).

According to another embodiment the present invention also relates to powders which contain (a) as active substance an antibody (b) as matrix forming agent a polyol, particularly mannitol. in a concentration of more than 20% (w/w), preferably from 25-60% (w/w), more preferably 25-50% (w/w), still more preferably 30-40% (w/w), and (c) an amino acid, preferably isoleucine, or a di- or tri-peptide, preferably with at least one isoleucine group such as tri-isoleucine, for example, in a concentration of 1-19.99% (w/w), and (d) the residual water content of said powder is below 1.2% (w/w), preferably below 1.0% (w/w). With the corresponding powder formulation it has surprisingly been possible to use polyols, particularly mannitol, in higher concentrations as the matrix-forming agent in the preparation of powders which contain antibodies as the pharmaceutical active substance, and thereby make use of the beneficial properties of the polyols, particularly mannitol. This was all the more surprising as proteins are normally destabilised by overdrying, i.e. by reducing the residual moisture content to less than 1% (w/w) (Breen et al., 2001, Pharm Res., 18(9), supra).

Preparation of the Powders According to the Invention

By freeze-drying it is possible to prepare powders whose residual moisture contents are <1% (w/w), and the same is also true of vacuum drying. These powders cannot be used for preparing formulations for inhalation as these processes do not produce inhalable particles. Spray drying offers an alternative which will produce sufficiently small particles. The residual moisture contents achieved are generally, however, significantly higher than with freeze-drying. This is particularly true of spray-dried powders which contain temperature-sensitive peptides/proteins as the pharmaceutical active substance. In particular antibodies or their derivatives can normally only be spray-dried at relatively low inlet temperatures (inlet temperature <150° C.).

Within the scope of the present invention it has surprisingly been found that efficient after-drying of spray-dried powders which contain sugar or polyols, preferably mannitol, as the matrix-forming agent in the range from 20-60% (w/w), are [sic] particularly stable on storage, particularly at temperatures above 20° C., and are characterised by high dispersibility. This applies particularly to the inhalable spray-dried powders. By special vacuum drying the residual moisture contents of the spray-dried powders were lowered to less than 1.2% (w/w), preferably to less than 1% (w/w), leading to a significantly improved protein stability and dispersibility. The improved stability of the proteins obtained by vacuum drying at 25-60° C., preferably at temperatures above 30° C. (32° C.), with a vacuum of 0.01-1 mbar, preferably 0.1 mbar, over 24 hours, was particularly surprising and could not have been foreseen. In fact, it is known from the literature that overdrying of proteins leads to a reduction in stability. Overdrying in this context means a reduction in the residual moisture content to less than 1% (cf. also Breen et al., 2001, supra). The powders with particular stability prepared by the present invention generally had residual moisture contents of less than 1% (w/w).

The present invention thus provides a process for preparing one of the spray-dried powders described above, characterised in that a spray solution containing at least one pharmaceutical active substance and a matrix-forming agent (a) is sprayed at a temperature below 200/120° C., preferably 150/70° C., and (b) the resulting powder is then dried in vacuo at a temperature of 25-60° C.

If other excipients, such as for example amino acids (isoleucine) or peptides (di- or tripeptides containing isoleucine), are present in addition to the pharmaceutical active substance and matrix forming agent, these are naturally also added to the spray solution. Preferably, the combinations of matrix forming agent and additional excipients described above are used to prepare a spray solution.

For this, the therapeutic active substance, preferably a biological macromolecule in the form of a peptide or protein, is dissolved in an aqueous solution, depending on the solubility conditions of the active substance in question. Usually, buffered solutions with a pH of 3-11, preferably 4-9 are used. When preparing inhalable powders an aqueous solution with a pH of 5.5-7.8 is particularly advantageous. In order to ensure sufficient solubility, the pH of the solution should be below the pI of the peptide/protein. The aqueous solution may optionally contain additional water-soluble organic solvents, such as e.g. acetone, alcohols or the like. Lower alcohols such as e.g. methanol, ethanol, propanol, (n or iso-propanol) or the like are particularly suitable. Mixed solvent systems of this kind normally contain between 0.1-80% (v/v), preferably between 10-40% (v/v), and particularly preferably between 10-20% (v/v) of a water-soluble organic solvent. The solid content in the solution to be sprayed is usually between 0.01-20% (w/v), preferably between 0.05-10% (w/v), particularly preferably between 0.1-2% (w/v). Within the scope of the present invention spray-dried powders were prepared starting from a solution with a solid content of 10% (w/v). Matrix forming agents and other excipients, if present, are dissolved together with the active substance or separately and sprayed.

The spraying is done in conventional spray driers, for example in apparatus made by Messrs Niro A/S (Soeborg, DK), Büchi Labortechnik GmbH (Flawil, CH) or the like. The optimum conditions for the spray drying depend in each case on the corresponding formulation and should be determined experimentally. The gas used is typically air, but inert gases such as nitrogen or argon are also suitable. In addition, the spray drying temperature, i.e. the inlet temperature and outlet temperature, is determined in accordance with the temperature sensitivity of the active substance used, in each case depending on the stabilisers used. An inlet temperature of 50-200° C. is usual, while the outlet temperature is usually 30-150° C. Within the scope of the present invention an inlet temperature of about 105° C. and an outlet temperature of 50-55° C. was used. However, rather higher temperatures are also possible, for example an inlet temperature of 120-200° C., preferably 90-130° C. and an outlet temperature of 70-120° C., preferably 50-80° C., depending on the amount of stabiliser. Spraying is generally carried out at a pressure of approximately 20-150 psi, preferably at about 30 or 40-100 psi, for example at about 30, 40, 50, 60, 70, 80, 90 or 100 psi.

The after-drying may be carried out particularly by drying in vacuo. This allows gentle drying at temperatures of 15-60° C., preferably between 20-40° C. In the present case a temperature above 30° C. was used. The optimum temperature in each case depends on the particular composition of the powder which is to be dried and the other drying conditions (e.g. drying time). A vacuum of 0.01-10 mbar, preferably 0.02-5 mbar, more preferably 0.02-1 mbar is suitable for the drying. It has proved particularly advantageous to carry out the drying at 0.05-0.5 mbar. The drying time was generally less than 30 hours, preferably about 24 hrs. Drying times of less than 24 hrs are also possible, for example 18, 15 or 12 hrs. However, drying at lower temperatures is also possible (approx. 20° C. or below). This usually presupposes a higher vacuum, however (=lower pressures). When drying powders containing an amount by weight of ≧30% of polyols, particularly mannitol, it has proved advantageous to select a drying temperature of about 25-35° C., a vacuum of 0.05-0.2 mbar and a drying time of 18-30 hrs. These conditions produced amorphous powders which were particularly stable on storage, containing a small proportion of aggregates, preferably <3.5%, even under extreme storage conditions of 15 weeks at 40° C. and 75% r.h.

Accordingly, in another embodiment the present invention relates to a process for preparing a spray-dried powder, characterised in that a spray solution containing at least one pharmaceutical active substance and a polyol, preferably mannitol, as matrix-forming agent, in a concentration of more than 20 but less than 60% (w/w) (a) is sprayed below a temperature of 105/60° C.; and (b) the resulting powder is then after-dried in vacuo at a pressure of about 0.05-0.2 mbar, a temperature of 25-60° C., preferably at 25-35° C. for about 18-30 hrs., preferably about 24 hrs. According to another embodiment, the matrix forming agent and pharmaceutical active substance are dissolved in separate spray solutions but sprayed together. At least one of the solutions to be sprayed may also contain other excipients such as, for example, amino acids, preferably isoleucine, peptidee in the form of tri- or di-peptides, preferably with an isoleucine group, such as tri-isoleucine. The spray solutions are of a nature such that spraying produces powders having the composition described in this patent specification.

Nature of the Powders According to the Invention

The powders of the present invention are characterised by their advantageous properties which consist of a constant good dispersibility, even after lengthy storage, a very good long-term stability and good inhalability into the lower respiratory tract. The latter is determined inter alia by the average particle size, the mean aerodynamic particle diameter (MMAD) and the amount of the so-called “Fine Particle Fraction” (FPF).

The particles herein preferably have a mean particle size of less than 20 μm, preferably less than 10 μm. According to a particularly preferred embodiment the particles according to the invention have a mean particle size of less than 7.5 μm, preferably of less than 5 μm. Particularly preferred are particles with a mean particle size of less than 4 μm and more preferably of less than 3.5 μm. In general the particles of the present invention have a mean particle diameter of 0.1-5 μm, preferably von 0.2-4 μm auf. In another embodiment the corresponding particles contain non-breathable particles, e.g. lactose, with a particle size of at least 40 μm, preferably between 40 and 200 μm.

Apart from the mean particle size the inhalability depends essentially on the mean aerodynamic particle diameter (MMAD). The particles according to the invention have an MMAD of less than 10 μm, preferably less than 7.5 μm. Particles with an MMAD of less than 5 μm, preferably less than 4 μm, even more preferably less than 3.5 μm are particularly advantageous. According to another preferred embodiment of the present invention the particles have an MMAD of less than 3 μm. In general the particles have an MMAD of 0.5-10 μm, preferably 0.5-7.5 μm, preferably 1-5 μm. The particles described in the Examples have a correspondingly advantageous mean aerodynamic particle diameter which is defined by the combination of optimum spray drying conditions and the choice and concentration according to the invention of the matrix-forming agent as well as other excipients, if present.

The particles according to the invention are also defined by their specific density. The particles according to the invention generally have a bulk density of 0.1-10 g/cm³, preferably 0.1-2 g/cm³. According to a particularly preferred embodiment the particles according to the invention have a bulk density of 1.1-1.5 g/cm³.

The particles according to the invention are particularly characterised by their very low residual moisture content in the powder, which accounts for the surprising properties of the particles according to the invention. After spray drying the particles according to the invention generally have a residual water content of up to 10% (w/w), preferably 2-9% (w/w). After subsequent after-drying, preferably in vacuo, the [noun missing] according to the invention have a residual water content of less than 1.2% (w/w), preferably less than 1.1% (w/w). Particularly preferred are particles with a residual water content of less than 1.0% (w/w), preferably of less than 0.9% (w/w), even more preferably less than 0.8% (w/w). In general, following after-drying, the powders according to the invention have a residual water content of 0.5-1.2% (w/w), preferably 0.6-1% (w/w), even more preferably 0.7-0.9% (w/w) auf. The particles contained in the powders according to the invention are predominantly hygroscopic. The low residual water content can be achieved by a combination of spray drying and after-drying in vacuo.

The particles according to the invention are characterised by their high, constant dispersibility. The spray drying conditions as well as the powder formulations used (e.g. the choice and concentration of the matrix-forming agent and any other excipients present) essentially determine the dispersibility. The particles according to the invention are characterised in that the percentage delivered is more than 30%, preferably more than 40%, and most preferably more than 50%. In a preferred embodiment the percentage delivered is more than 60%, preferably more than 70%, particularly preferably more than 75%, even more preferably more than 80%. The powder preparations described in the Examples had a delivery percentage of between 60-90%. Powders with a mannitol content of 30 to 40% (w/w) and a residual water content of less than 1.2% (w/w), preferably less than 1% (w/w), more preferably less than 0.9% (w/w) or even 0.8% (w/w) proved to be particularly dispersible. The percentage delivered, measured using a Handihaler®, was more than 80%.

Another yardstick for measuring the good performance of the particles is the proportion of the “Fine particles Fraction” in the powder preparations. The powder preparations according to the invention according to another embodiment of the invention have a proportion of FPF_{(7.5)}, in other words a proportion of particles with an MMAD of less than 7.5 μm, of at least 35%, preferably at least 40%, more preferably at least 45%, even more preferably at least 50%, e.g. 55%, 60%, 65%, etc. According to another preferred embodiment of the present invention the powder preparations have [with] a proportion of FPF_{(3.5)}, in other words a proportion of particles with an MMAD of 3.5-0.5 μm, of at least 30%, preferably at least 35%, for example at least 40, 45, 50, 55, 60%. This size distribution is particularly suitable for inhalation deep into the respiratory tract.

The powder preparations obtained by the invention are also characterised by very good long-term stability, which means the performance of the powders remains constant over a number of weeks under extreme storage conditions. The surprisingly good physical and chemical stability of the powders contributes to this. The proportion of pharmaceutical active substance present in aggregated form in the powder is less than 3.5% for the particles according to the invention, preferably less than 3.2% in relation to the total quantity of active substance contained in the powder. According to a preferred embodiment the amount of aggregated active substance is less than 3.0%, preferably less than 2.8%, for example less than 2.7, 2.6, 2.5, 2,4, 2,3, 2.2, 2.1, 2.0, 1.9, 1.8%, etc, again based on the total quantity of active substance contained in the powder. This includes storage for 4, in some cases 15, weeks at 30 or 40° C. and at a relative humidity of 60 or 75%. Accordingly the proportion of free monomeric active substance is typically more than 95%, preferably more than 96.5%, more preferably more than 96.8%, for example 97.0, 97.1, 97.2, 97,3, 97,4, 97.5, 97.6, 97.7, 97.8, 97.9, 98.0, 98.1, 98.2% etc, again based on the total quantity of active substance contained in the powder. The powders prepared in the Examples, particularly those with a polyol content of 20-40% (w/w), particularly 30-40% (w/w), had a content of non-aggregated antibody of more than 96.5% (proportion of aggregates was approx. 3.2-3.3%) after 15 weeks' storage at a relative humidity of 75%.

Administration of the Powders According to the Invention

Basically, the powder preparations according to the invention may be administered directly as dry powders using so-called dry powder inhalers, or after reconstitution in the form of aerosols using so-called nebulisers. The inhalable powders according to the invention may be administered using inhalers known from the prior art.

Inhalable powders according to the invention may be administered, for example, by means of inhalers which deliver a single dose from a supply using a measuring chamber as described in U.S. Pat. No. 4,570,630A, or by other means as described in DE 36 25 685 A. Preferably, the inhalable powders according to the invention are packed into capsules (to produce so-called inhalettes) which are used in inhalers as described, for example, in WO 94/28958.

Other examples of suitable inhalers may be found inter alia in U.S. Pat. No. 5,458,135; U.S. Pat. No. 5,785,049 or WO 01/00263. Other suitable inhalers are known from WO 97/41031; U.S. Pat. No. 3,906,950 and U.S. Pat. No. 4,013,075. Other dispersion inhalers for dry powder preparations are described in EP 129 985; EP 472 598; EP 467 172 and U.S. Pat. No. 5,522,385.

The inhalable powders according to the invention may for example be administered using the inhaler known by the name Turbuhaler® (AstraZeneca LP) or with inhalers as disclosed for example in EP 237 507 A. Other suitable inhalers are the Rotahaler® or the Discus® (both made by GlaxoSmithKline Corp.), the Spiros™ inhaler (Dura Pharmaceuticals) and the Spinhaler® (Fiscon).

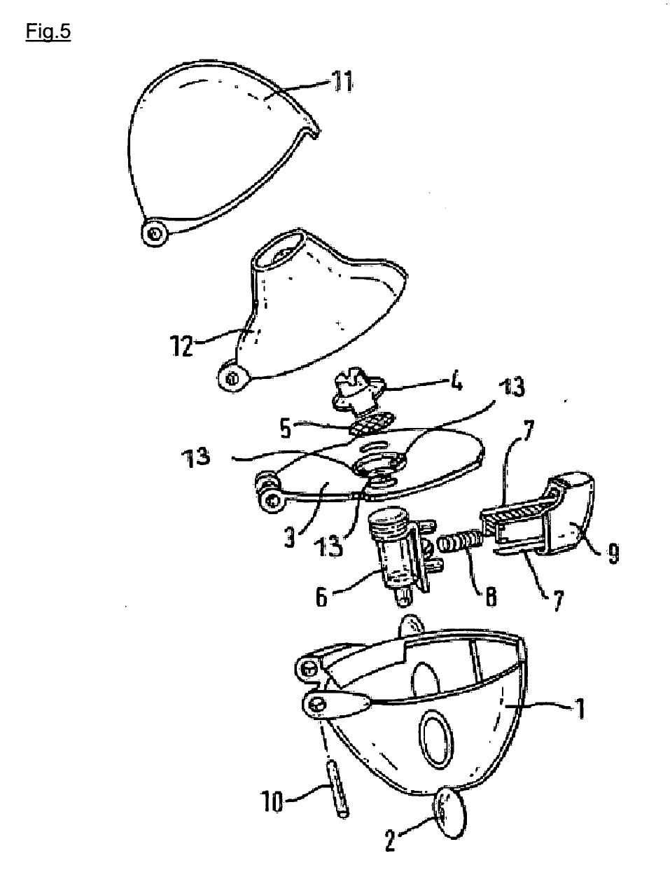

A particularly preferred inhaler for administering the pharmaceutical combination in inhalettes according to the invention is shown in FIG. 5. This inhaler (Handyhaler) for inhaling powdered pharmaceutical compositions from capsules is characterised by a housing 1 containing two windows 2, a deck 3 in which there are air inlet ports and which is provided with a screen 5 secured via a screen housing 4, an inhalation chamber 6 connected to the deck 3 on which there is a push button 9 provided with two sharpened pins 7 and movable counter to a spring 8, and a mouthpiece 12 which is connected to the housing 1, the deck 3 and a cover 11 via a spindle 10 to enable it to be flipped open or shut, as well as air through-holes 13 for adjusting the flow resistance.

If the inhalable powders according to the invention are to be packed into capsules (inhalettes) for the preferred use described above, the quantities packed into each capsule should be 1 to 30 mg.

The powders according to the invention may also be administered as propellant-containing inhalable aerosols. For this, the powders according to the invention are reconstituted in an aqueous solution. Suitable solutions are known in the art. For example, it is advantageous to reconstitute the powders in physiological solutions with a pH of 3-11, preferably 4-9. Reconstitution in an aqueous solution with a pH of 5.5-7.8 is particularly advantageous. The solution for reconstituting the powders according to the invention may also contain further excipients in the form of stabilisers, emulsifiers, surfactants or water-soluble organic solvents. Corresponding substances are known to the skilled man and described for example in Bauer, Lehrbuch der Pharmazeutischen Technologie, Wissenschaftl. Verlagsgesellschaft mbH, Stuttgart, 178-184; Adler, 1998, Journal of Pharmaceutical Sciences, 88(2), 199-208. Corresponding inhalable aerosols which are prepared by reconstituting the powders according to the invention are also a subject of the present invention.

The propellant gases which may be used to prepare the inhalation aerosols according to the invention are also known from the prior art. Suitable propellant gases are selected from among hydrocarbons such as n-propane, n-butane or isobutane and halohydrocarbons such as preferably chlorinated and fluorinated derivatives of methane, ethane, propane, butane, cyclopropane or cyclobutane. The propellant gases mentioned above may be used on their own or in mixtures thereof. Particularly preferred propellant gases are halogenated alkane derivatives selected from TG11, TG12, TG134a (1,1,1,2-tetrafluoroethane), TG227 (1,1,1,2,3,3,3-heptafluoropropane) and mixtures thereof, the propellant gases TG134a, TG227 and mixtures thereof being preferred.

The inhalation aerosols containing propellant gas according to the invention may contain up to 5% (w/w) of active substance. Aerosols according to the invention contain, for example, 0.002 to 5 wt.-%, 0.01 to 3 wt.-%, 0.015 to 2 wt.-%, 0.1 to 2 wt.-%, 0.5 to 2 wt.-% or 0.5 to 1 wt.-% of the pharmaceutical active substance. Inhalable aerosols with an active substance concentration in this range by may prepared by controlled reconstitution of the powders according to the invention in a corresponding amount of solvent.

The propellant-driven inhalation aerosols according to the invention mentioned above may be administered using inhalers known in the art (MDIs=metered dose inhalers). Reference may be made here to the Ventolin® (Ventolin Pharmacy) or the inhalers described in U.S. Pat. No. 5,32,094 or U.S. Pat. No. 5,672,581. Accordingly, in another aspect, the present invention relates to pharmaceutical compositions in the form of propellant-driven aerosols as hereinbefore described combined with one or more inhalers suitable for administering these aerosols. In addition, the present invention relates to inhalers which are characterised in that they contain the propellant gas-containing aerosols described above according to the invention.

The present invention also relates to cartridges which are fitted with a suitable valve and can be used in a suitable inhaler and which contain one of the above-mentioned propellant gas-containing inhalation aerosols according to the invention. Suitable cartridges and methods of filling these cartridges with the inhalable aerosols containing propellant gas according to the invention are known from the prior art.

The powders according to the invention may also be reconstituted in propellant-free inhalable solutions or suspensions. Corresponding propellant-free inhalable solutions contain for example aqueous or alcoholic, preferably ethanolic solvents, optionally ethanolic solvents mixed with aqueous solvents. In the case of aqueous/ethanolic solvent mixtures the relative proportion of ethanol compared with water is not limited but the maximum is preferably up to 70 percent by volume, more particularly up to 60 percent by volume of ethanol. The remainder of the volume is made up of water. Co-solvents and/or other excipients as described above may be added to the propellant-free inhalable solutions according to the invention. Preferred co-solvents are those which contain hydroxyl groups or other polar groups, e.g. alcohols—particularly isopropyl alcohol, glycols—particularly propyleneglycol, polyethyleneglycol, polypropyleneglycol, glycolether, glycerol, polyoxyethylene alcohols and polyoxyethylene fatty acid esters. The terms excipients and additives in this context denote any pharmacologically acceptable substance which is not an active substance but which can be formulated with the active substance or substances in the pharmacologically suitable solvent in order to improve the qualitative properties of the active substance formulation. Preferably, these substances have no pharmacological effect or, in connection with the desired therapy, no appreciable or at least no undesirable pharmacological effect. The excipients and additives include, in addition to those described above, for example, surfactants such as soya lecithin, oleic acid, sorbitan esters, such as polysorbates, polyvinylpyrrolidone, other stabilisers, complexing agents, antioxidants and/or preservatives which guarantee or prolong the shelf life of the finished pharmaceutical formulation, flavourings, vitamins and/or other additives known in the art. The additives also include pharmacologically acceptable salts such as sodium chloride as isotonic agents. The preferred excipients include antioxidants such as ascorbic acid, for example, provided that it has not already been used to adjust the pH, vitamin A, vitamin E, tocopherols and similar vitamins and provitamins occurring in the human body. Preservatives may be used to protect the formulation from contamination with pathogens. Suitable preservatives are those which are known in the art, particularly cetyl pyridinium chloride, benzalkonium chloride or benzoic acid or benzoates such as sodium benzoate in the concentration known from the prior art. The preservatives mentioned above are preferably present in concentrations of up to 50 mg/100 ml, more preferably between 5 and 20 mg/100 ml. Accordingly, the present invention also includes propellant-free inhalable aerosols which are prepared by reconstituting the powders according to the invention.

The propellant-free inhalable solutions according to the invention are administered in particular using inhalers of the kind which are capable of nebulising a small amount of a liquid formulation in the therapeutic dose within a few seconds to produce an aerosol suitable for therapeutic inhalation. Within the scope of the present invention, preferred inhalers are those in which a quantity of less than 100 μL, preferably less than 50 μL, more preferably between 10 and 30 μL of active substance solution can be nebulised in preferably one spray action to form an aerosol with an average particle size of less than 20 μm, preferably less than 10 μm, such that the inhalable part of the aerosol corresponds to the therapeutically effective quantity.

An apparatus of this kind for propellant-free delivery of a metered quantity of a liquid pharmaceutical composition for inhalation is described for example in International Patent Application WO 91/14468 and also in WO 97/12687 (cf. in particular FIGS. 6a and 6b). Reference is specifically made within the scope of the present invention to the corresponding FIGS. 6a and 6b of WO 97/12687 including the associated parts of the description. The nebulisers (devices) described therein are also known by the name Respimat® (Boehringer Ingelheim Pharma). Because of its cylindrical shape and handy size of less than 9 to 15 cm long and 2 to 4 cm wide, this device can be carried at all times by the patient. The nebuliser sprays a defined volume of the pharmaceutical formulation using high pressures through small nozzles so as to produce inhalable aerosols.

The preferred atomiser essentially consists of an upper housing part, a pump housing, a nozzle, a locking mechanism, a spring housing, a spring and a storage container, characterised by
a pump housing which is secured in the upper housing part and which comprises at one end a nozzle body with the nozzle or nozzle arrangement,
a hollow plunger with valve body,
a power takeoff flange in which the hollow plunger is secured and which is located in the upper housing part,
a locking mechanism situated in the upper housing part,
a spring housing with the spring contained therein, which is rotatably mounted on the upper housing part by means of a rotary bearing,
a lower housing part which is fitted onto the spring housing in the axial direction.

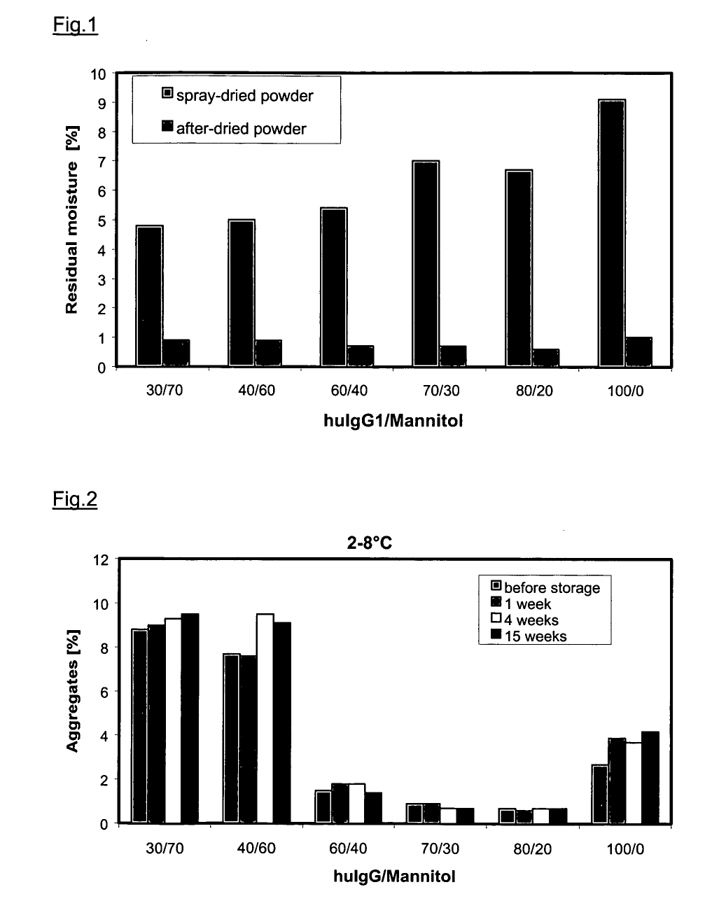

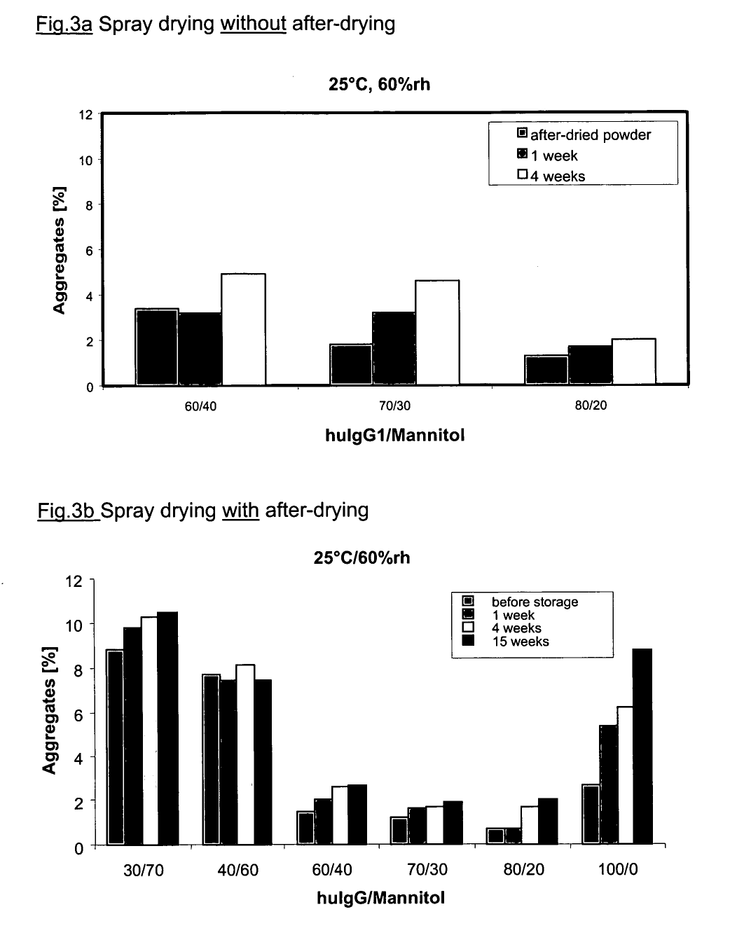

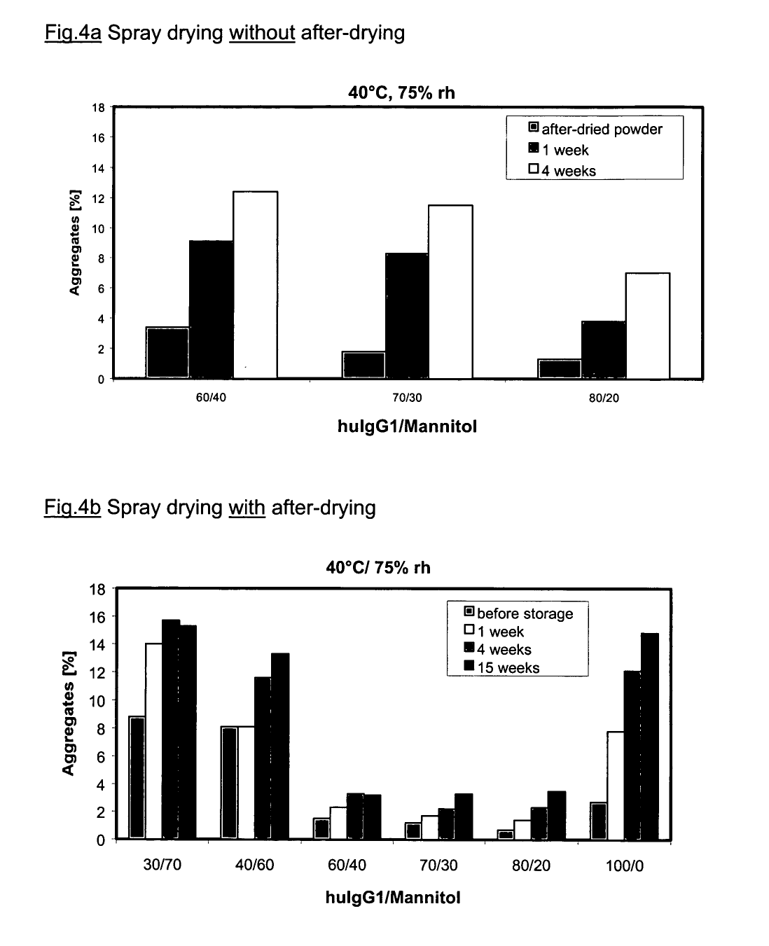

The hollow plunger with valve body corresponds to a device disclosed in WO 97/12687. It projects partially into the cylinder of the pump housing and is axially movable within the cylinder. Reference is made in particular to FIGS. 1 to 4, especially FIG. 3, and the relevant parts of the description. The hollow plunger with valve body exerts a pressure of 5 to 60 MPa (about 50 to 600 bar), preferably 10 to 60 MPa (about 100 to 600 bar) on the fluid, the measured amount of active substance solution, at its high pressure end at the moment when the spring is actuated. Volumes of 10 to 50 microlitres are preferred, while volumes of 10 to 20 microlitres are particularly preferred and a volume of 15 microlitres per spray is most particularly preferred.

The valve body is preferably mounted at the end of the hollow plunger facing the valve body.

The nozzle in the nozzle body is preferably microstructured, i.e. produced by microtechnology. Microstructured nozzle bodies are disclosed for example in WO-94/07607; reference is hereby made to the contents of this specification, particularly FIG. 1 disclosed therein and the associated description. The nozzle body consists for example of two sheets of glass and/or silicon firmly joined together, at least one of which has one or more microstructured channels which connect the nozzle inlet end to the nozzle outlet end. At the nozzle outlet end there is at least one round or non-round opening 2 to 10 microns deep and 5 to 15 microns wide, the depth preferably being 4.5 to 6.5 microns while the length is preferably 7 to 9 microns. In the case of a plurality of nozzle openings, preferably two, the directions of spraying of the nozzles in the nozzle body may extend parallel to one another or may be inclined relative to one another in the direction of the nozzle opening. In a nozzle body with at least two nozzle openings at the outlet end the directions of spraying may be inclined at an angle of 20 to 160° to one another, preferably 60 to 150°, most preferably 80 to 100°. The nozzle openings are preferably arranged at a spacing of 10 to 200 microns, more preferably at a spacing of 10 to 100 microns, most preferably 30 to 70 microns. Spacings of 50 microns are most preferred.

The directions of spraying will therefore meet in the vicinity of the nozzle openings.

The liquid pharmaceutical preparation strikes the nozzle body with an entry pressure of up to 600 bar, preferably 200 to 300 bar, and is atomised into an inhalable aerosol through the nozzle openings. The preferred particle or droplet sizes of the aerosol are up to 20 microns, preferably 3 to 10 microns.

The locking mechanism contains a spring, preferably a cylindrical helical compression spring, as a store for the mechanical energy. The spring acts on the power takeoff flange as an actuating member the movement of which is determined by the position of a locking member. The travel of the power takeoff flange is precisely limited by an upper and lower stop. The spring is preferably biased, via a power step-up gear, e.g. a helical thrust gear, by an external torque which is produced when the upper housing part is rotated counter to the spring housing in the lower housing part. In this case, the upper housing part and the power takeoff flange have a single or multiple V-shaped gear.

The locking member with engaging locking surfaces is arranged in a ring around the power takeoff flange. It consists, for example, of a ring of plastic or metal which is inherently radially elastically deformable. The ring is arranged in a plane at right angles to the atomiser axis. After the biasing of the spring, the locking surfaces of the locking member move into the path of the power takeoff flange and prevent the spring from relaxing. The locking member is actuated by means of a button. The actuating button is connected or coupled to the locking member. In order to actuate the locking mechanism, the actuating button is moved parallel to the annular plane, preferably into the atomiser; this causes the deformable ring to deform in the annular plane. Details of the construction of the locking mechanism are given in WO 97/20590.

The lower housing part is pushed axially over the spring housing and covers the mounting, the drive of the spindle and the storage container for the fluid.

When the atomiser is actuated the upper housing part is rotated relative to the lower housing part, the lower housing part taking the spring housing with it. The spring is thereby compressed and biased by means of the helical thrust gear and the locking mechanism engages automatically. The angle of rotation is preferably a whole-number fraction of 360 degrees, e.g. 180 degrees. At the same time as the spring is biased, the power takeoff part in the upper housing part is moved along by a given distance, the hollow plunger is withdrawn inside the cylinder in the pump housing, as a result of which some of the fluid is sucked out of the storage container and into the high pressure chamber in front of the nozzle.

If desired, a number of exchangeable storage containers which contain the fluid to be atomised may be pushed into the atomiser one after another and used in succession. The storage container contains the aqueous aerosol preparation according to the invention.

The atomising process is initiated by gently pressing the actuating button. As a result, the locking mechanism opens up the path for the power takeoff member. The biased spring pushes the plunger into the cylinder of the pump housing. The fluid leaves the nozzle of the atomiser in atomised form.

Further details of construction are disclosed in PCT Applications WO 97/12683 and WO 97/20590, to the contents of which reference is hereby made.

The components of the atomiser (nebuliser) are made of a material which is suitable for its purpose. The housing of the atomiser and, if its operation permits, other parts as well are preferably made of plastics, e.g. by injection moulding. For medicinal purposes, physiologically safe materials are used.

FIGS. 6 a/b of WO 97/12687, including the associated description to which reference is hereby made once more, show a corresponding nebuliser (Respimat®). This is particularly suitable for administering the propellant-free inhalable aerosols according to the invention.

FIG. 6 a of WO 97/12687 shows a longitudinal section through the atomiser with the spring under tension, FIG. 6 b of WO 97/12687 shows a longitudinal section through the atomiser with the spring released. The upper housing part (51) contains the pump housing (52), on the end of which is mounted the holder (53) for the atomiser nozzle. In the holder is the nozzle body (54) and a filter (55). The hollow piston (57) fixed in the power take-off flange (56) of the locking clamping mechanism projects partly into the cylinder of the pump housing. At its end the hollow piston carries the valve body (58). The hollow piston is sealed off by the gasket (59). Inside the upper housing part is the stop (60) on which the power take-off flange rests when the spring is relaxed. Located on the power take-off flange is the stop (61) on which the power take-off flange rests when the spring is under tension. After the tensioning of the spring, the locking member (62) slides between the stop (61) and a support (63) in the upper housing part. The actuating button (64) is connected to the locking member. The upper housing part ends in the mouthpiece (65) and is closed off by the removable protective cap (66). The spring housing (67) with compression spring (68) is rotatably mounted on the upper housing part by means of the snap-fit lugs (69) and rotary bearings. The lower housing part (70) is pushed over the spring housing. Inside the spring housing is the replaceable storage container (71) for the fluid (72) which is to be atomised. The storage container is closed off by the stopper (73), through which the hollow piston projects into the storage container and dips its end into the fluid (supply of active substance solution). The spindle (74) for the mechanical counter is mounted on the outside of the spring housing. The drive pinion (75) is located at the end of the spindle facing the upper housing part. On the spindle is the slider (76).

If the formulation according to the invention is nebulised using the method described above (Respimat®), the mass expelled, in at least 97%, preferably at least 98% of all the actuations of the inhaler (puffs), should correspond to a defined quantity with a range of tolerance of not more than 25%, preferably 20% of this quantity. Preferably, between 5 and 30 mg, more preferably between 5 and 20 mg of formulation are delivered as a defined mass per puff.

However, the formulation according to the invention can also be nebulised using inhalers other than those described above, for example jet-stream inhalers or other stationary nebulisers.

Accordingly, in another aspect, the present invention relates to pharmaceutical compositions in the form of propellant-free inhalable solutions or suspensions as hereinbefore described in conjunction with a device suitable for administering these formulations, preferably in conjunction with the Respimat®. Preferably the present invention is directed to propellant-free Inhalable solutions or suspensions, containing one of the powders according to the invention, in conjunction with the device known as a Respimat®. Moreover the present invention relates to the above-mentioned devices for inhalation, preferably the Respimat®, characterised in that they contain the propellant-free inhalable solutions or suspensions according to the invention as described above.

According to the invention, inhalable solutions containing one of the powders according to the invention as described herein in a single preparation are preferred.

The propellant-free inhalable solutions or suspensions according to the invention may take the form of concentrates or sterile inhalable solutions or suspensions ready for use, as well as the above-mentioned solutions and suspensions designed for use in the Respimat®. Formulations ready for use may be produced from the concentrates, for example, by the addition of isotonic saline solutions. Sterile formulations ready for use may be administered using energy-operated fixed or portable nebulisers which produce inhalable aerosols by means of ultrasound or compressed air by the Venturi principle or other principles.

Accordingly, in another aspect, the present invention relates to pharmaceutical compositions in the form of propellant-free inhalable solutions or suspensions as described hereinbefore which take the form of concentrates or sterile formulations ready for use, combined with a device suitable for administering these solutions, characterised in that the device is an energy-operated free-standing or portable nebuliser which produces inhalable aerosols by means of ultrasound or compressed air by the Venturi principle or other methods.

Other suitable nebulisers for inhaling reconstituted aerosols are the AERx™ (Aradigm), Ultravent® (Mallinkrodt) and AconII® (Maquest Medical Products).

### EXAMPLES

The Examples which follow serve to illustrate the present invention in more detail without restricting the scope of the invention to the following embodiments by way of example.

Materials:

IgG1 is a humanised monoclonal antibody with a molecular weight of about 148 kDa. The antibody is derived from a murine antibody in which the complementarity-determining regions of the murine antibody have been transferred to a human immunoglobulin structure. A chimeric antibody has been produced with 95% human content and 5% murine content. The antibody is expressed by murine myeloma cell lines. The cells are removed by Tangential Flow Microfiltration and the cell-free solution is purified by various chromatographic methods. Other steps include nuclease treatment, treatment at a low pH and nanofiltration. The bulk solution contains histidine and glycine as buffer and has been concentrated, for the preparation of the solution for spray drying, by diafiltration to approx. 100 mg/ml. The bulk for the preparation of the spray solution contained 0.6% aggregates. The antibody is commercially available as a sterile lyophilised product for intramuscular administration. The finished drug can be stored at 2-8° C. for at least 2 years.

Spray Drying with Büchi B-290:

The prepared solutions consisting of IgG1/mannitol were spray-dried using a Büchi Mini Spray Dryer B-290 made by Messrs Büchi Labortechnik (AG, CH).

The spray drier is made up of a heating system, a filter, an aspirator, a drying tower, a cyclone, temperature sensors for measuring the inlet and outlet temperature and a collecting vessel. The solution to be sprayed is pumped into the two-substance nozzle by means of a peristaltic pump. There, the solution is atomised into small drops which are dried in the spray tower. The drying in the spray tower is done using heated air which is aspirated through the spray tower by the direct current method by means of the aspirator. The product is collected in the collecting vessel after passing through the cyclone.

The solid content of the spray solutions was 10% (w/V) in 100 ml of sprayed solution. The inlet temperature was 90° C., the liquid feed rate approx. 3 ml/min, the aspirator flow rate 35 m³/h and the atomiser flow rate 0.67 m³/h. This produced an outlet temperature of 50-55° C.

Size Exclusion Chromatography (SEC-HPLC):

Size exclusion chromatography was used to determine soluble aggregates in the spray solutions and in the reconstituted powders. The SEC-HPLC was carried out with a HP1090 made by Messrs Agilent (Waldbronn, Del.). The column used was a TSK3000SWXL column (300×7.8 mm) made by Messrs Tosoh Biosep (Stuttgart, Del.). The buffer consisting of 0.1M di-sodium hydrogen phosphate-dihydrate, 0.1M sodium sulphate was dewatered and adjusted to pH 6.8 with ortho-phosphoric acid 85%. The amount of sample put in was 25 μl at a protein concentration of between 2-10 mg/ml. The protein was detected using a diode-array detector at 280 nm. The chromatographs were evaluated using the Chemstation software made by Agilent.

Vacuum Driving:

The vacuum drying was carried out in a vacuum drying cupboard. The temperature was adjusted to 32° C., the vacuum was adjusted to about 0.1 mbar. The samples were put into in 2R vials to dry in the cupboard, with the stoppers removed completely and also left to dry in the cupboard. The drying time was 24 h.

Test to Determine the Stability on Storage:

After the spray drying the samples were stored in climate-controlled cupboards. The conditions were 2-8° C., 25° C./60% r.h., 40° C./75% r.h. The humidity levels were adjusted using corresponding saturated solutions, i.e. sodium chloride for 75% r.h. and ammonium nitrate for 60% r.h. The samples were stored open in some cases, but also surrounded by a border and heat-sealed in aluminium containers. Samples were taken after 3 days, 1, 4, 15 and 52 weeks. After 3 days only open samples were taken out, and after 1 and 4 weeks both open and closed samples were taken, and after 15 and 52 weeks only closed samples were taken. The subsequent analysis for investigation for aggregates was carried out with SEC-HPLC.

Determining the Water Content According to Karl-Fischer:

The residual water content in the dried products was determined by coulometric titration (Metrohm 737 KF Coulometer with 703 titration level, Filderstadt, Del.). For the measurement powdered methanol (Hydranal—methanol dry, VWR/Merck Eurolab, Darmstadt, Del.) was dissolved or dispersed. The measuring solution (Hydranal—Coulomat solution, VWR/Merck Eurolab, Darmstadt, Del.) of the Metrohm coulometer was conditioned at the start of the measurements, i.e. the measuring solution was titrated to a zero water content. The sample was injected into the titration cell.

X-Ray Diffractometry (Wide-Angle X-ray Diffractometry (WAXS)):

In order to determine the crystallinity of the dried samples the samples were investigated with a Seifert X-ray diffractometer XRD 3000 TT (Messrs Seifert, Ahrensburg, Del.) in a chamber at a controlled temperature of 22° C. The X-ray tube Cu anode, Cu—Kα-radiation with λ=0.15418 mm (Ni primary filter), was operated at an anode voltage of 40 kV and a current strength of 30 mA. After the sample dish had been placed in the apparatus the sample was measured in the range from 5 to 40° at a scan rate of 2θ=0.05° with 2 sec measuring time at each angle.

The powder diffractograms were taken with the ScanX-Rayflex application, Version 3.07 device XRD 3000 (Scan), or the Rayflex Version 2.1, 1996 (Analyse) on the SC 1000 V detector.

Determining the Mean Particle Size of the Particles:

The mean particle sizes of the particles was determined using the Sympatech Helos made by Messrs Sympatech GmbH (Clausthal-Zellerfeld, Del.). The measuring principle is based on laser diffraction, using a helium neon laser. 1-3 mg of powder are dispersed with an air pressure of 2 bar, and passed through a parallel laser beam in front of the Fourier lens (50 mm). The particle size distribution is evaluated using a Fraunhofer model.

Determining the Aerodynamic Diameter of the Particles:

The aerodynamic particle diameter was determined using the APS 3321 (Aerodynamic Particle Sizer) of Messrs TSI GmbH, Particle Instruments (Aachen, Del.). The underlying measuring principle is a Time-of-Flight measurement in which the particles are accelerated through a nozzle. For this, the formulations are packed into gelatine capsules and introduced into the apparatus using the Handihalerg, in a flow volume of 39 ml/min which is produced by a vacuum pump, and deposited on the baffle plate and filter (delivery time 6.15s, Delta P Aerosol apparatus set to approx. 0.47 inch W.C., and to between 0.2-0.8 inches W.C during the delivery).

Determining the Amount Delivered:

The amount delivered is determined using the APS 3321 Aerodynamic Particle Sizer of Messrs TSI GmbH, Particle Instruments (Aachen, Del.). The powder is delivered in a flow volume of 39 ml/min by the Handihaler, and is deposited on the baffle plate and on a filter. The capsules are weighed after the delivery and from this the amount delivered is determined. The amount delivered is obtained as the difference in the weight of the powder, minus the residue of powder remaining in the capsule after delivery.

### Example 1

### 30/70 huIgG1/Mannitol for Pulmonary Use

The bulk of the antibody was mixed with mannitol in a corresponding ratio and spray-dried. A 30% huIgG1 solution with 70% mannitol was obtained by topping up 27.5 ml of bulk solution (c=109 mg/ml) with 7.0 g mannitol up to 100 ml of water. The moisture content of the formulation directly after spray drying was 4.8% and was lowered to 0.9% by subsequent vacuum after-drying. 50 mg of powder was dissolved in 5 ml of water for SEC-HPLC. The amount of aggregate, determined by HPSEC, was 5.1% after spray drying and 8.8% after the subsequent after-drying. On storing at 2-8° C. the amount of aggregate after 4 weeks was 9.3% and after 15 weeks it was 9.5%. The amount of aggregate increased to 10.3% after a storage period of 4 weeks at 25° C./60% r.h., and to 10.5% after 15 weeks. The aggregate content was 15.7% after storage for 4 weeks at 40° C./75% r.h., and 15.3% after 15 weeks.

### Example 2

### 40/60 huIgG1/Mannitol for Pulmonary Use

The bulk of the antibody was mixed with mannitol in a corresponding ratio and spray-dried by the method described. A 40% huIgG1 solution with 60% mannitol was obtained by topping up 36.7 ml of bulk solution (c=109 mg/ml) with 6.0 g mannitol up to 100 ml of water. The moisture content of the formulation directly after spray drying was 5.0% and was lowered to 0.9% by subsequent vacuum after-drying. 50 mg of powder was dissolved in 5 ml of water for SEC-HPLC. The amount of aggregate, determined by HPSEC, was 1.1% after spray drying and 1.5% after the subsequent after-drying. On storing at 2-8° C. the amount of aggregate after 4 weeks was 9.5% and after 15 weeks it was 9.1%. The amount of aggregate increased to 8.1% after a storage period of 4 weeks at 25° C./60% r.h., and to 7.4% after 15 weeks. The aggregate content was 11.6% after storage for 4 weeks at 40° C./75% r.h., and 13.3% after 15 weeks. The MMAD was 7.4 μm and the amount delivered was 74.1%.

### Example 3

### 60/40 huIgG1/Mannitol for Pulmonary Use

The bulk of the antibody was mixed with mannitol in a corresponding ratio and spray-dried by the method described. A 60% huIgG1 solution with 40% mannitol was obtained by topping up 55.1 ml of bulk solution (c=109 mg/ml) with 4.0 g mannitol up to 100 ml of water. The moisture content of the formulation directly after spray drying was 5.4% and was lowered to 0.7% by subsequent vacuum after-drying. 50 mg of powder was dissolved in 5 ml of water for SEC-HPLC. The amount of aggregate was 1.1% after spray drying and 1.5% after the subsequent after-drying. On storing at 2-8° C. the amount of aggregate after 4 weeks was 1.8% and after 15 weeks it was 1.4%. The amount of aggregate increased to 2.5% after a storage period of 4 weeks at 25° C./60% r.h., and to 2.7% after 15 weeks. The aggregate content was 3.3% after storage for 4 weeks at 40° C./75% r.h., and 3.2% after 15 weeks. The MMAD was 7.5 μm and the amount delivered was 85.7%.

### Example 4

### 70/30 huIgG1/Mannitol for Pulmonary Use

The bulk of the antibody was mixed with mannitol in a corresponding ratio and spray-dried by the method described. A 70% huIgG1 solution with 30% mannitol was obtained by topping up 64.2 ml of bulk solution (c=109 mg/ml) with 3.0 g mannitol up to 100 ml of water. The moisture content of the formulation directly after spray drying was 7.0% and was lowered to 0.7% by subsequent vacuum after-drying. 50 mg of powder was dissolved in 5 ml of water for SEC-HPLC. The amount of aggregate was 0.6% after spray drying and 0.9% after the subsequent after-drying. On storing at 2-8° C. the amount of aggregate after 4 weeks was 0.7% and after 15 weeks it was 0.7%. The amount of aggregate increased to 1.7% after a storage period of 4 weeks at 25° C./60% r.h., and to 1.9% after 15 weeks. The aggregate content was 2.2% after storage for 4 weeks at 40° C./75% r.h., and 3.3% after 15 weeks. The MMAD was 5.7 μm and the amount delivered was 80.2%.

### Example 5

### 80/20 huIgG1/Mannitol for Pulmonary Use

The bulk of the antibody was mixed with mannitol in a corresponding ratio and spray-dried by the method described. An 80% huIgG1 solution with 20% mannitol was obtained by topping up 73.4 ml of bulk solution (c=109 mg/ml) with 2.0 g mannitol up to 100 ml of water. The moisture content of the formulation directly after spray drying was 6.7% and was lowered to 0.6% by subsequent after-drying. The amount of aggregate was 0.6% after spray drying and 0.7% following the after-drying. 50 mg of powder was dissolved in 5 ml of water for SEC-HPLC. On storing at 2-8° C. the amount of aggregate after 4 weeks was 0.7% and after 15 weeks it was 0.7%. The amount of aggregate increased to 1.7% after a storage period of 4 weeks at 25° C./60% r.h., and to 2.0% after 15 weeks. The aggregate content was 2.3% after storage for 4 weeks at 40° C./75% r.h., and 3.5% after 15 weeks. The MMAD was 5.5 μm and the amount delivered was 72.4%.

### Example 6

### 100/0 huIgG1/Mannitol for Pulmonary Use

The bulk of the antibody was spray-dried without the addition of an excipient by the method described. The moisture content of the formulation directly after spray drying was 9.1% and was lowered to 1.0% by subsequent vacuum after-drying. 50 mg of powder was dissolved in 5 ml of water for SEC-HPLC. The amount of [sic] On storing at 2-8° C. the amount of aggregate after 4 weeks was 3.7% and after 15 weeks it was 4.2%. aggregate was 2.7% after spray drying and 2.7% after the subsequent after-drying. The amount of aggregate increased to 6.1% after a storage period of 4 weeks at 25° C./60% r.h., and to 8.7% after 15 weeks. The aggregate content was [sic]% after storage for 4 weeks at 40° C./75% r.h., and 12.1% after 15 weeks.

### Example 7

### Preparation of Other Powders According to the Invention

60/40 huIgG1/Mannitol for Pulmonary Use

The bulk of the antibody is combined with mannitol in a suitable ratio and spray-dried by the method described here and then dried. A 60% huIgG1 solution containing 40% mannitol is obtained by mixing 13.76 ml of bulk solution (c=109 mg/ml) with 1.0 g mannitol and topping up to 100 ml with water. The solids content of the solution is 2.5% (w/V).

70/30 huIgG1/Mannitol for Pulmonary Use

The bulk of the antibody is combined with mannitol in a suitable ratio and spray-dried by the method described here and then dried. A 70% huIgG1 solution containing 30% mannitol is obtained by combining 16.05 ml of bulk solution (c=109 mg/ml) with 0.75 g mannitol and topping up to 100 ml with water. The solids content of the solution is 2.5% (w/V).

70/25/5 huIgG1/Mannitol/Isoleucine for Pulmonary Use

The bulk of the antibody is combined with mannitol and isoleucine in a suitable ratio and spray-dried by the method described here and then dried. A 70% huIgG1 solution containing 25% mannitol and 5% isoleucine is obtained by combining 16.05 ml bulk solution (c=109 mg/ml) with 0.625 g mannitol and 0.125 g isoleucine and topping up to 100 ml with water. The solids content of the solution is 2.5% (w/V).

60/30/10 huIgG1/Mannitol/Isoleucine for Pulmonary Use

The bulk of the antibody is combined with mannitol and isoleucine in a suitable ratio and spray-dried by the method described here and then dried. A 60% huIgG1 solution containing 30% mannitol and 10% isoleucine is obtained by combining 13.76 ml bulk solution (c=109 mg/ml) with 0.75 g mannitol and 0.25 g isoleucine and topping up to 100 ml with water. The solids content of the solution is 2.5% (w/V).

70/25/5 huIgG1/Mannitol/Tri-Isoleucine for Pulmonary Use

The bulk of the antibody is combined with mannitol and tri-isoleucine in a suitable ratio and spray-dried by the method described here and then dried. A 70% huIgG1 solution containing 25% mannitol and 5% tri-isoleucine is obtained by combining 16.05 ml of bulk solution (c=109 mg/ml) with 0.625 g mannitol and 0.125 g of tri-isoleucine and topping up to 100 ml with water. The solids content of the solution is 2.5% (w/V).

60/30/10 huIgG1/Mannitol/Tri-Isoleucine for Pulmonary Use

The bulk of the antibody is combined with mannitol and tri-isoleucine in a suitable ratio and spray-dried by the method described here and then dried. A 60% huIgG1 solution containing 30% mannitol and 10% tri-isoleucine is obtained by combining 13.76 ml of bulk solution (c=109 mg/ml) with 0.75 g mannitol and 0.25 g tri-isoleucine and topping up to 100 ml with water. The solids content of the solution is 2.5% (w/V).

1. A spray-dried amorphous powder comprising a pharmaceutical active substance and a matrix-forming agent, wherein the powder has a residual moisture content of less than 1.2% (w/w).

2. The spray-dried amorphous powder according to claim 1, wherein the powder has a residual moisture content of less than 1% (w/w).

3. The spray-dried powder according to claim 1, wherein the powder consists predominantly of finely divided inhalable particles with an MMAD and/or MMD of less than 10 μm.

4. The spray-dried powder according to claim 3, wherein the powder consists predominantly of finely divided inhalable particles with an MMAD and/or MMD between 0.5 and 7.5 μm.

5. The spray-dried powder according to claim 3, wherein the powder consists predominantly of finely divided inhalable particles with an MMAD and/or MMD between 1 and 5 μm

6. The spray-dried powder according to claim 1, wherein the matrix-forming agent is selected from a sugar, polyol, polymer or a combination thereof.

7. The spray-dried powder according to claim 3, wherein the matrix-forming agent is selected from a sugar, polyol, polymer or a combination thereof.

8. The spray-dried powder according to claim 6, wherein the sugar is selected from a mono-, di-, oligo- or polysaccharide or a combination thereof.

9. The spray-dried powder according to claim 7, wherein the sugar is selected from a mono-, di-, oligo- or polysaccharide or a combination thereof.

10. The spray-dried powder according to claim 6, wherein the polyol is mannitol.

11. The spray-dried powder according to claim 7, wherein the polyol is mannitol.

12. The spray-dried powder according to claim 10, wherein the proportion of mannitol is between 20 and 60% (w/w) of the dry mass of the powder.

13. The spray-dried powder according to claim 11, wherein the proportion of mannitol is between 20 and 60% (w/w) of the dry mass of the powder.

14. The spray-dried powder according to claim 10, wherein the proportion of mannitol is between 25 and 50% (w/w) of the dry mass of the powder.

15. The spray-dried powder according to claim 11, wherein the proportion of mannitol is between 25 and 50% (w/w) of the dry mass of the powder.

16. The spray-dried powder according to claim 10, wherein the proportion of mannitol is between 30 and 40% (w/w) of the dry mass of the powder.

17. The spray-dried powder according to claim 11, wherein the proportion of mannitol is between 30 and 40% (w/w) of the dry mass of the powder.

18. The spray-dried powder according to claim 1, wherein the powder further comprises an additional excipient.

19. The spray-dried powder according to claim 3, wherein the powder further comprises an additional excipient.

20. The spray-dried powder according to claim 18, wherein the additional excipient is an amino acid, preferably isoleucine, a peptide, a surfactant and/or a salt.

21. The spray-dried powder according to claim 19, wherein the additional excipient is an amino acid, preferably isoleucine, a peptide, a surfactant and/or a salt.

22. The spray-dried powder according to claim 20, wherein the peptide is one or more di- and/or tri-peptides, preferably one or more di- and/or tri-peptides with one or more isoleucine groups.

23. The spray-dried powder according to claim 21, wherein the peptide is one or more di- and/or tri-peptides, preferably one or more di- and/or tri-peptides with one or more isoleucine groups.

24. The spray-dried powder according to claim 22, wherein the peptide is tri-isoleucine.

25. The spray-dried powder according to claim 23, wherein the peptide is tri-isoleucine.

26. The spray-dried powder according to claim 1, wherein the pharmaceutical active substance is a biological macromolecule.

27. The spray-dried powder according to claim 3, wherein the pharmaceutical active substance is a biological macromolecule.

28. The spray-dried powder according to claim 26, wherein the biological macromolecule is a polypeptide or protein.

29. The spray-dried powder according to claim 27, wherein the biological macromolecule is a polypeptide or protein.

30. The spray-dried powder according to claim 28, wherein the polypeptide or protein is a growth factor, an enzyme or an antibody.

31. The spray-dried powder according to claim 29, wherein the polypeptide or protein is a growth factor, an enzyme or an antibody.

32. The spray-dried powder according to claim 30, wherein the proportion of growth factors, enzymes or antibodies present in the powder in aggregated form is less than 3.5% (w/w).

33. The spray-dried powder according to claim 31, wherein the proportion of growth factors, enzymes or antibodies present in the powder in aggregated form is less than 3.5% (w/w).

34. A pharmaceutical composition comprising a spray-dried powder according to claim 1.

35. A pharmaceutical composition comprising a spray-dried powder according to claim 3.

36. The pharmaceutical composition according to claim 34 for administration by inhalation, wherein the pharmaceutical composition is in the form of a propellant-containing metered-dose aerosol or a propellant-free inhalable solution.

37. The pharmaceutical composition according to claim 35 for administration by inhalation, wherein the pharmaceutical composition is in the form of a propellant-containing metered-dose aerosol or a propellant-free inhalable solution.

38. A process for preparing a spray-dried powder according to claim 1, wherein a spray solution containing at least one pharmaceutical active substance and a matrix-forming agent
a) is sprayed below a temperature of 200/120° C. (inflow/outflow temperature); and
b) the resulting powder is then after-dried in vacuum at a temperature above 15° C. and below 60° C.

39. The process according to claim 38, wherein the after-drying is carried out at a pressure of between 0.01 and 10 mbar.

40. The process according to claim 38 or 39, wherein the spray-dried powder is after-dried for at least 12 hrs.

41. A Process for preparing a spray-dried powder according to claim 1, wherein a spray solution containing at least one pharmaceutical active substance and a matrix-forming agent
a) is sprayed below a temperature of 105/60° C.; and
b) the resulting powder is then after-dried in vacuum at a pressure of about 0.05 to about 0.2 mbar, at a temperature above 30° C. and below 60° C., for about 18 to about 30 hrs.

42. An inhalable spray-dried powder comprising:
a) a mannitol content of ≧20% (w/w);
b) antibodies as the pharmaceutically active substance; and
c) a residual moisture content of ≦1.2% (w/w).
